(19) United States
(12) Patent Application Publication
(10) Pub. No.: US 2023/0029081 A1
(43) Pub. Date: Jan. 26, 2023

(54) APPLICATORS AND METHODS FOR APPLYING A MICRONEEDLE PATCH TO A SKIN OF A SUBJECT, AND MICRONEEDLE PATCHES

(71) Applicant: UPRAX MICROSOLUTIONS B.V., JD Delft (NL)

(72) Inventors: Bernardus Hendrikus VAN OORSCHOT, JD Delft (NL); Koen VAN DER MAADEN, JD Delft (NL)

(21) Appl. No.: 17/786,503

(22) PCT Filed: Dec. 17, 2020
(86) PCT No.: PCT/NL2020/050800

§ 371 Date: Jun. 16, 2022

(30) Foreign Application Priority Data
Dec. 18, 2019 (EP) PCT/EP2019/086126

(51) Int. Cl. A61M 37/00

(52) U.S. Cl. CPC A61M 37/0015; A61M 2037/0023; A61M 2037/0061

(57) ABSTRACT

A reusable applicator for applying a microneedle patch to a skin of a subject which comprises a base for positioning onto the skin. The base comprises a skin-side end and a holder for holding the microneedle patch in position relative to the skin-side end. Two or more contact parts at the skin-side end can contact the skin. One or more are movable over the skin and away from the other contact 5 parts to stretch the skin at least during penetration of the skin by a microneedle of the patch. The applicator further comprises an interface for an actuator. The actuator actuates in operation a movement of the microneedle patch relative to the skin-side end to penetrate at least into the stratum corneum of the epidermis of the skin with the microneedle. A microneedle patch comprises a skin-adhesive surface for attaching the patch to the skin of a10 subject. The patch has one or more microneedles projecting from the skin-adhesive surface out of the patch. A stiffening body stiffens the patch in at least a parallel direction parallel to the skin-adhesive surface in a region of the skin-adhesive surface which includes the microneedle.

### FIELD OF THE INVENTION

This invention relates to applicators and methods for applying a microneedle patch to a skin of a subject, and microneedle patches.

### BACKGROUND OF THE INVENTION

A common technique for delivering drugs from a subject across a biological barrier is the use of a hypodermic needle, such as those used with standard syringes or catheters, to transport drugs across (through) the skin. While effective for this purpose, hypodermic needles generally cause pain; local damage to the skin at the site of insertion; bleeding, which increases the risk of disease transmission; and a wound sufficiently large to be a site of infection. The withdrawal of bodily fluids or other samples, such as for diagnostic purposes, using a conventional hypodermic needle has these same disadvantages. Hypodermic needle techniques also generally require administration by one trained in its use. The needle technique also is undesirable for long term, controlled continuous drug delivery.

Another delivery technique is the transdermal patch, which usually relies on diffusion of the drug across the skin. However, this method is not useful for many drugs, due to the poor permeability (i.e. effective barrier properties) of the skin. The rate of diffusion depends in part on the size and hydrophilicity of the drug molecules and the concentration gradient across the stratum corneum. Few drugs have the necessary physiochemical properties to be effectively delivered through the skin by passive diffusion. Iontophoresis, electroporation, ultrasound, and heat (so-called active systems) have been used in an attempt to improve the rate of delivery. While providing varying degrees of enhancement, these techniques are not suitable for all types of drugs, failing to provide the desired level of delivery. In some cases, they are also painful and inconvenient or impractical for continuous controlled drug delivery over a period of hours or days. Attempts have been made to design alternative devices for active transfer of drugs, or analyte to be measured, through the skin.

As an alternative transdermal delivery technique, microneedle patches have been developed. Microneedle patches are patches with, very small, structures, typically shorter than 1 mm, which can be pressed onto the skin of a subject and pierce the skin, see e.g. McConville, Aaron et al. “Mini-Review: Assessing the Potential Impact of Microneedle Technologies on Home Healthcare Applications.” Medicines (Basel, Switzerland) vol. 5,2 50. 8 Jun. 2018, incorporated herein by reference. Through the pierced skin, drugs or other substances may then be delivered into the body of the subject, or alternatively samples be taken from the body.

Although various types of applicators are known, such as from European Patent EP 2 906 285, up to now their performance is unsatisfactory for many applications.

Furthermore, a general problem of microneedle patches is that the effectiveness of the microneedle is typically impacted by the relatively uncontrolled conditions under which they are applied and maintained applied on the skin.

### SUMMARY OF THE INVENTION

The present invention provides in a first aspect applicators and methods of applying a microneedle patches with applicators as described in the accompanying claims.

The present invention provides in a second aspect microneedle patches and methods of applying such microneedle patches as described in the accompanying claims.

The present invention provides in a third aspect kits comprising applicators and microneedle patches as described in the accompanying claims.

Specific embodiments of the invention are set forth in the dependent claims.

These and other aspects of the invention will be apparent from and elucidated with reference to the examples described hereinafter.

### BRIEF DESCRIPTION OF THE DRAWINGS

Further details, aspects and embodiments of the invention will be described, by way of example only, with reference to the drawings. In the drawings, like reference numbers are used to identify like or functionally similar elements. Elements in the figures are illustrated for simplicity and clarity and have not necessarily been drawn to scale.

FIG. 1 schematically shows a perspective view of a first example of an embodiment of an applicator for a microneedle patch.

FIG. 2 schematically shows a cross-sectional view of the example of FIG. 1, taken along the line I-I in FIG. 1.

FIGS. 3-5 schematically show cross-sectional views of alternatives to aspects of the example of FIGS. 1 and 2.

FIGS. 6-14 schematically show cross-sectional views similar to FIG. 2, of the example of FIG. 1 in a method of applying a microneedle patch on the skin of subject.

FIG. 15-16 schematically show cross-sectional views similar to FIG. 2 of other examples of embodiments of an applicator for a microneedle patch.

FIG. 17-18 schematically show cross-sectional views similar to FIG. 2 of yet another example of an embodiment of an applicator for a microneedle patch.

FIG. 19-20 schematically show perspective views of a further example of an embodiment of an applicator for a microneedle patch.

FIG. 21 schematically shows a cross-sectional view of the example of FIG.19-20, taken along the line II-II in FIG. 19.

FIG. 22-26 schematically cross-sectional views similar to FIG. 21, of the example of FIGS. 19 and 20 in a method of applying a microneedle patch on the skin of subject.

FIG. 27 schematically a cross-sectional view of yet another example of an embodiment of an applicator for a microneedle patch.

FIG. 28-30 schematically cross-sectional views of the example of FIG. 27 in a method of applying a microneedle patch on the skin of subject.

FIG. 31 schematically a cross-sectional view of a variant of the example of FIG. 27.

FIG. 32 shows a top view of an example of a microneedle patch.

FIG. 33 shows cross-sectional views taken along the line III-III in FIG. 32 of the example of FIG. 31 in an unapplied state (A) and an applied state (B).

FIG. 34 shows cross-sectional views of examples of patches in a state applied on the skin of a subject.

FIG. 35 shows a combined top and bottom view of another example of a microneedle patch.

FIG. 36 shows a cross-sectional view taken along the line IV-IV in FIG. 35.

FIG. 37 schematically shows a sectional view of another example of a microneedle patch.

FIG. 38 schematically shows a top view of an example of a packaging of patches suitable for a kit with an applicator.

FIG. 39 schematically shows a sectional view of an example of a kit of a patch and an applicator.

### DETAILED DESCRIPTION OF THE PREFERRED EMBODIMENTS

### Applicator

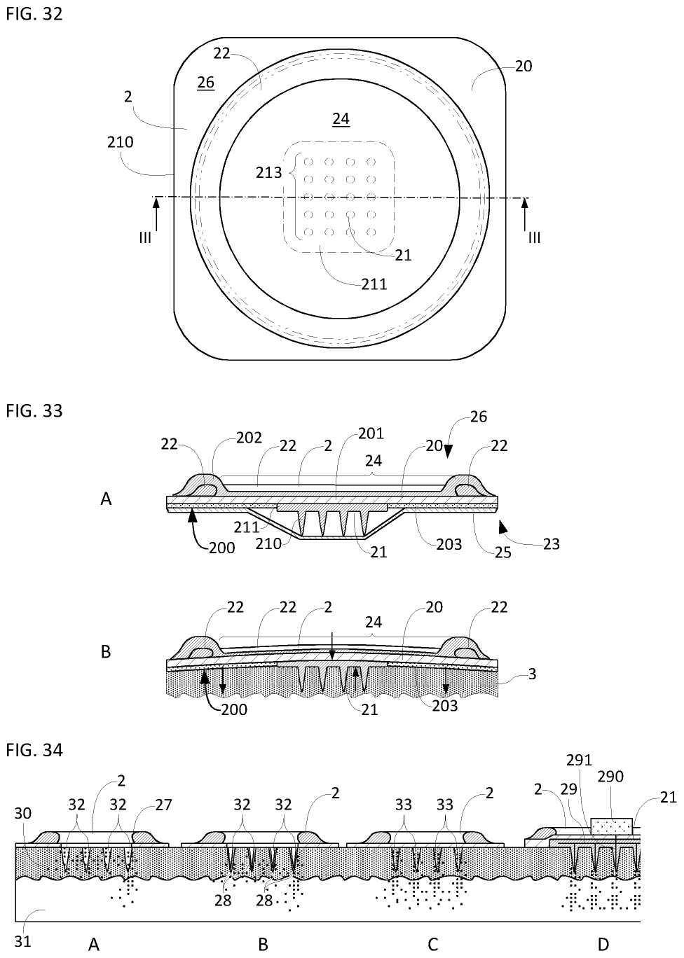

In the following, examples of applicators for microneedle patches are described which can be used to stretch the skin, and to generate a movement of the microneedle patch relative to the skin to penetrate at least into, or through, the stratum corneum of the epidermis of the stretched skin with the microneedle. More specific, when the skin is penetrated, one or more microneedles of the microneedle patch create respective perforations in the respective layers. Via the perforations, operations between the skin and the patch may be performed. For example, a pharmaceutically active substance may be administered from the patch to the subject and/or through the microneedles a substance may be collected from the subject, e.g. dermally or transdermally. Furthermore, e.g. characteristics of the penetrated layers or below may be modified with the patch, e.g. by heating the perforated area with the microneedle, or properties of the skin be sensed to name a couple of examples. The microneedle patch can e.g. be a patch as described below in the section “Patch” with reference to the examples of FIGS. 32-37, but may alternatively be another type of patch, with one or more, e.g. an array, of microneedles.

By stretching the skin, the skin is tightened and depression of the skin by the microneedle(s) under the force exerted by the microneedle patch in the contact area is at least partially inhibited. Thus, the microneedle will easier penetrate the skin. Also, the actual depth of the perforations in the skin can be closer to the length the microneedles project out of the patch, and accordingly for the same desired depth of perforation in the skin shorter microneedles may be used. This can reduce the risk that the microneedles activate the nociceptors in the skin of the subject, and accordingly allows to reduce unpleasant sensations experienced by the subject upon or after application of the patch.

In addition, in case the skin is maintained stretched after perforation, the perforations made by the microneedle in the skin can be kept open, and accordingly the exchange of substances between the subject and the microneedle patch can be improved. This further enables an improved control of the properties of the perforations, and render them less dependent on the body part and/or subject specific characteristics of the skin.

Also, in case the skin is maintained stretched after perforation, the microneedle can be better maintained in the perforations, and the risk that the skin pushes the microneedle out of the perforation may be reduced.

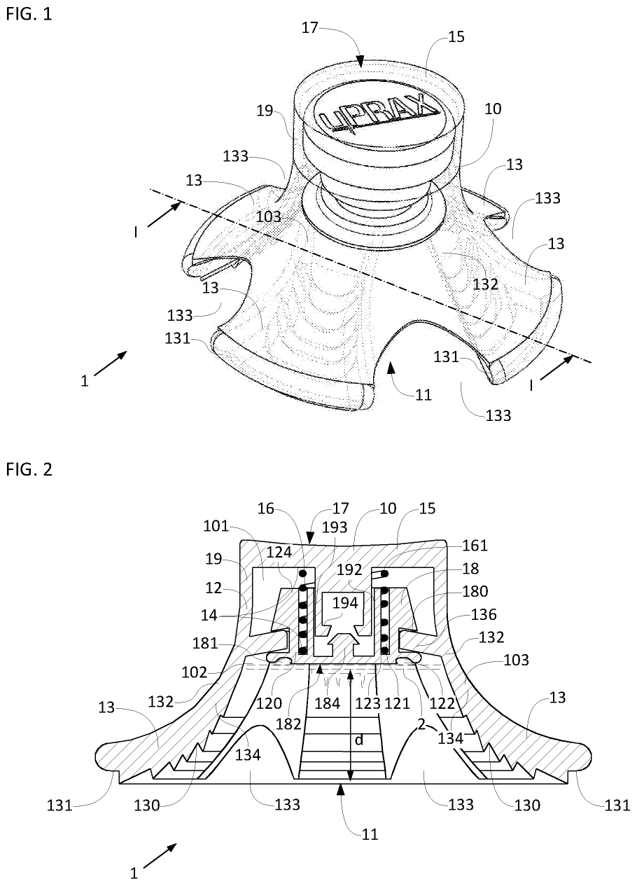

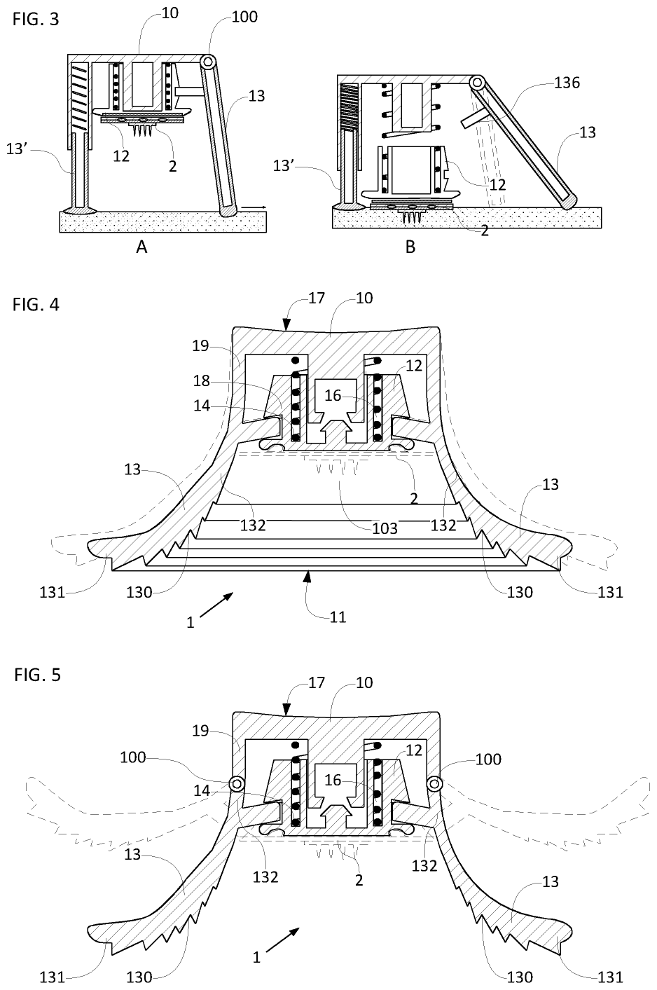

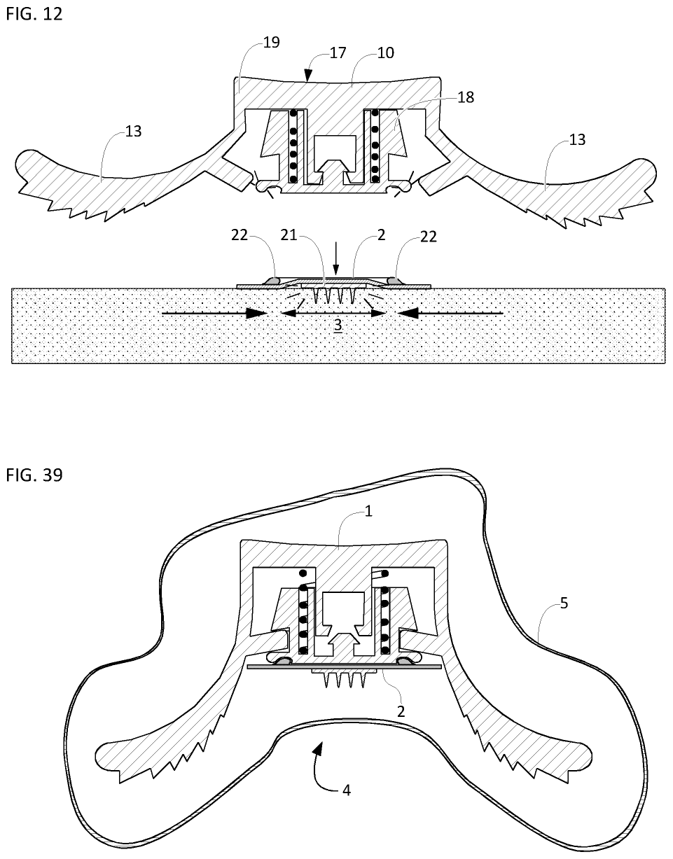

Referring to FIGS. 1 and 2, the first example of an applicator 1 shown therein comprises a base 10 which can be positioned onto the skin 3 (shown in FIGS. 3-14 and not shown in FIGS. 1-2) of a subject, e.g. a living human being. The base 10 comprises a bottom or skin-side end 11, and in this example has a top 15 comprising a contact surface 17 for a hand of the human operator and the distal-end opposite of the skin-side end 11. When the applicator 1 is placed correctly, the skin-side end 11 contacts the skin 3 whereas the top 15 does not contact the skin 3.

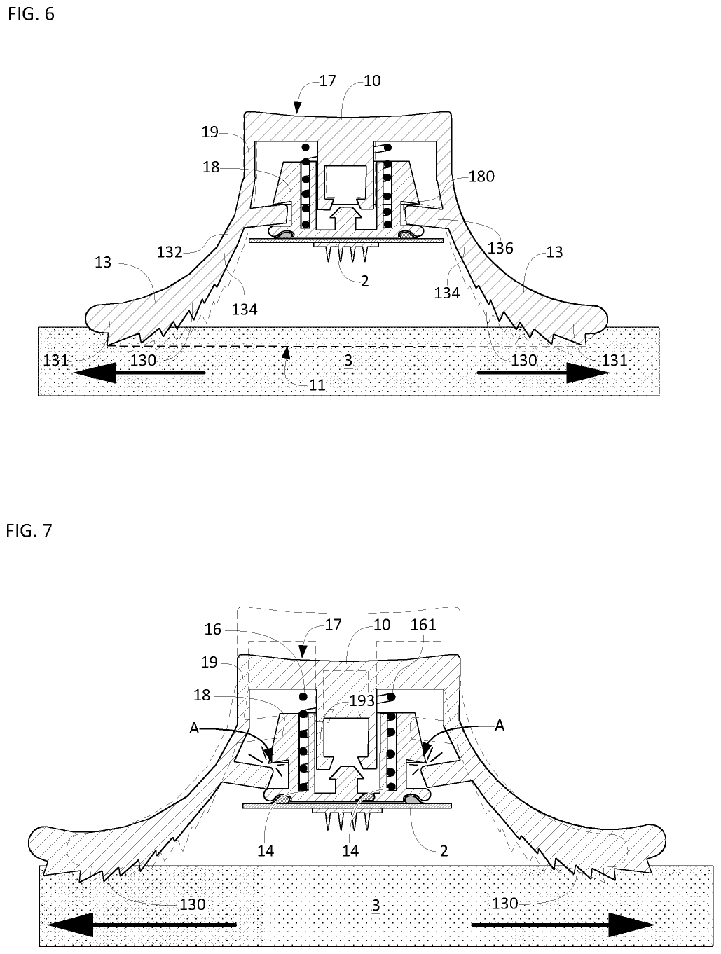

The base 10 further comprises a holder 12 for a microneedle patch 2 (which is not present in FIGS. 1 and 2 but the position of which is indicated with the dashed lines). The base 10 further comprises skin contact parts 13, in this example leg-shaped, at the skin-side end 11 for contacting the skin. In this example, the base has four separate contact parts 13, but it will be apparent that the base may generally have two, three, or more than four contact parts. As illustrated in more detail in FIGS. 6-14, one, two or more of the contact parts 13 are movable over the skin 3, and away from other contact parts 13, to stretch the skin at least during penetration of the skin by the microneedle. The applicator 1 further comprises an interface 14 for an actuator 16. As illustrated in more detail in FIGS. 6-14, the actuatorl6 actuates, when in operation, a movement of the microneedle patch 2 relative to the skin 3 to penetrate at least into, or through, the stratum corneum of the epidermis of the skin with the microneedle.

Referring to FIGS. 1 and 2, the first example of an applicator 1 shown therein can be used to apply a microneedle patch 2 to a skin 3 of a subject, as explained in more detail with reference to FIGS. 6-18. The microneedle patch 2 can be applied by positioning the applicator 1 provided with the microneedle patch 2 on the skin 3. The skin 3 may be stretched with the moving contact part(s) 13 and the movement of the microneedle patch 2 towards to the skin 3 may be actuated. The skin 3 is then penetrated with the microneedle 21, by the movement of the microneedle patch 2. The microneedle 21 penetrates in at least into, and preferably through, the stratum corneum and optionally, further into the epidermis. In this respect, the microneedles may pierce completely through a layer or penetrate into the layer without piercing through the layer. The microneedles may for example penetrate deeper into the skin, and pierce through the epidermis, until into the dermis or into the hypoderm is subcutis. The microneedles can for example penetrate the dermis until into the papillary dermis or until into the reticular dermis. The microneedles can e.g. pierce the stratum corneum, and any intermediate layers, until into one of the following skin layers without piercing that layer: stratum lucidum, stratum granulosum, stratum spinonsum, stratum basale, basement membrane, papillary dermis, reticular dermis. Preferably, but not necessarily, the penetration of the microneedle(s) avoids activation of the nociceptors in the skin. After penetration, substances can be exchanged between the microneedle patch 2 and the body of the subject though the perforated area of the skin 3. This exchange can e.g. be transdermal or dermal. For example, pharmaceutically active ingredients be administered through the perforations, samples be taken from the subject through the perforations, or the microneedles be used as, or connect to, sensors for measuring properties of the body of the subject, or other operations be performed with the microneedle on the body of the subject.

The holder 12 may be implemented in any manner suitable for the specific implementation. When the microneedle patch 2 is present in the holder 12, the holder 12 holds in this example the microneedle patch 2 at a distance (as indicated with the arrow d between the dashed line and the patch 2) from the skin-side end 11. Thus, in this example, when the applicator 1 is placed with the skin-side end 11 on the skin 3, the patch 2 is at a distance from the skin 3. The distance allows to accelerate the microneedle patch 2 when applying the patch, such that the impact of the patch 2 (together with the holder) on the skin 3 is sufficient to penetrate the stretched skin 3.

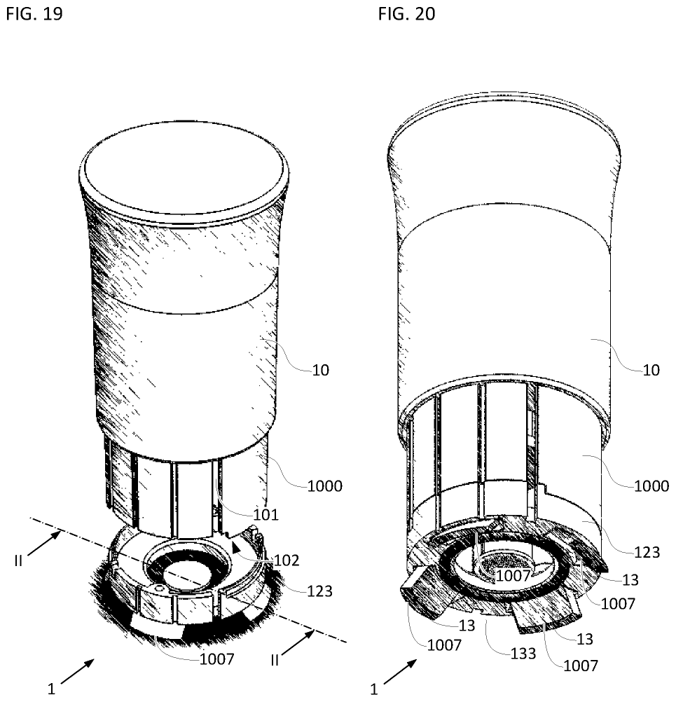

However, depending on the specific implementation and patch, the holder 12 may be implemented to hold the patch 2 positioned at the skin-side end 11 such that the microneedles contact the skin 3 when the applicator 1 is placed, such as shown in FIG. 22 for the example of FIG. 19-21, or projecting beyond the skin-side end 11 to push into the skin when the applicator 1 is placed. In such a case, for example, an impact may be generated on a distal, impact surface of the holder, which is transferred onto the microneedle patch contacting the skin 3 and which drives the microneedles into the skin.

In the example of FIGS. 1-2 the holder 12 comprises a movable platform 18 which is movable relative to the skin-side end 11, in a direction towards the skin 3 between an initial position, in which the skin is not perforated by the microneedles, and a perforating position in which the microneedles penetrate the skin. The holder 12 further comprises a base-body 19 for holding the movable platform 18 in position relative to the base-body 19. In the examples, the platform 18 is releasably fixated to the base-body 19. The base-body in this example holds the platform 18 suspended above the skin until the platform 18 is released to move, relative to the base-body, towards the skin, as explained further down below.

Although the base-body 19 can hold the platform 18 in a larger variety of manners, in this example the platform 18 is releasably fixated inside the base-body 19. In this example, the base 10 comprises a space 101 which has an opening 102 facing the skin-side end 11 through which the platform 18 can pass. On the platform 18 the microneedle patch can be releasably mounted. The platform is in this example part of an insert 123 and located at a skin-side of the insert 123. The insert 123 has a proximal side 124 admitted into the space 101.

The insert 123 is interlocked with the base-body 19. Thus, the movement of the base-body 19 towards the skin-side end 11 causes a movement in the same direction of the platform 18, and in this example the transmission ratio is 1:1. That is moving the base-body over a distance X causes a movement in the same direction of the platform over the same distance X, as long as the platform 18 and the base-body are interlocked.

More specifically, the base-body 19 has in this example a hollow-shape, and in this example the space 101 is formed by a recess or blind-hole which has an open-side 102 facing towards the skin-side end 11. The platform 18 is admitted in this recess, with the microneedle patch 2, when placed on the platform, facing the skin-side end 11 while the path between the patch 2 and the skin-side end 11 is unobstructed, or can be free-ed, to allow the patch 2 to move from the initial position towards, e.g. up to or beyond, the skin-side end 11.

In this example the blind-hole is relatively large and the base-body 19 has an open bottom and a closed top. Although other shapes are possible, in this example the base-body 19 is cup-shaped with an upside-down orientation. It will be apparent that e.g. the not-filled parts of the space 101 not occupied by the platform 18 may be filled, and thus the base-body be a solid body with a relatively small bore for example, and that a large variety of other shapes is possible. Although other shapes are possible, in this example the base-body 19 has a cylindrical outer and inner shape. The base-body is shaped and dimensioned to fit into a hand of a user. At the top 15, the outer surface of the base-body 19 is flat, with in this example a slightly concave shape to provide a contour complementary to the hand of a user, e.g. a medical practitioner or the subject, but which may e.g. be planar or convex.

Seen from the top 15, the first, hollow, part of the base-body 19 in which the platform 18 is admitted, transitions into a skirt or collar 103 which projects, seen in direction from the top 15 to the skin-side end 11, beyond the location of the patch 2. The collar 103 thus protects or shields in this example the patch 2 prior to application on the skin 3 against mechanical contact, and thus allows to avoid e.g. inadvertent damage to the microneedles 21 and/of contamination of the microneedles with micro-organisms due to contact with unsterile surfaces.

In this example, the collar 103 extents up to the skin-side end 11, but alternatively e.g. separate legs may be provided between the collar and the skin-side end 11. In this example, the skirt widens towards the skin-side end 11, which reduces the risk that the movement of the patch 2 during application is hampered by the base-body 18. In this example, the skirt has trumpet-like shape and flares towards the skin-side end 11, but the skirt 103 may alternatively have a frusto-conical shape or not widen at all and have e.g. a cylindrical shape.

As shown, between the location of the patch 2 and the skin side end 11, the skirt transitions into the contact parts 13, and to that end is at the skin-side end 11 provided with cut-outs 133 extending from the skin-side end 11 of the skirt towards the position of the patch 2. In this example the cut-outs 133 extend from the skin-side end 11 up to an upper end of the cut-out, which lies below the position of the patch 2, but alternatively some or all of the cut-outs may extend up to the patch, or higher e.g. up to or close to the top 15. These cut-outs thus form gaps between the contact parts 13. As illustrated in FIG. 4, alternatively, the skirt may be without such cut-outs and the skin-side end 11 be formed by an uninterrupted “seam” of the skirt and the contact parts 13 thus be different parts of an integral element, e.g. a monolithic element. In such a case, the skirt may e.g. by of a material which is elastically stretchable in the radial direction of the skirt, perpendicular to the axial direction from the top 15 to the skin-side end 11. In addition, or alternatively, the gap between some or all of the contact parts 13 can be partially, or entirely be filled with a thinner, different or the same as the material of the contact parts 13, material or another material with is more elastic than the contact parts 13. This provides a closed inside of the basse while less force is required to stretch the skin 3 and move the top 15 towards the skin-side end 11.

Furthermore, depending on the specific implementation, for instance the contact parts 13 may be provided with reinforcing ribs at the inside or outside, e.g. which extend in the direction from the top 15 to the skin-side end 11 at the inwards and/or outwards facing surface of the contact parts 13.

On the platform 18, the microneedle patch 2 may be placed oriented with a skin-adhesive surface 200 facing the skin-side end 11. To that end, the platform 18 may comprise, as in this example, a releasable, form or force closable, connector 122 for reliably attaching the patch 2 on the platform 18. The releasable connector 122 can e.g. be releasable by, upon or after penetration, moving the platform 18 away from the skin 3. In this example, the releasable connector 122 comprises a releasable clamp for reliably clamping the patch on the platform, and thus forming a form closed connection. To that end, as more clearly shown in FIG. 11, the releasable clamp may comprise a female part, such as a recess in a contact surface 182 of the platform 18 which contacts the patch 2. In the female, in an initial, unused state of the patch, a plastically or elastically deformable male part of the patch 2 may be jammed. In response to moving the platform 18 away from the skin 3 after the skin-adhesive surface of the patch 2 has been attached to the skin, the jammed part may then pop out of the recess.

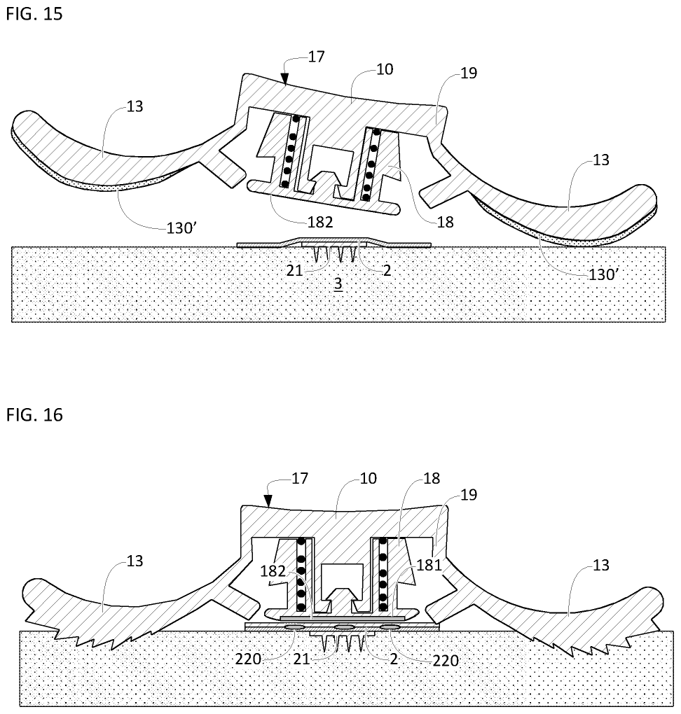

However, other releasable attachments may likewise be used, and for example the patch 2 can be attached with a low tack pressure-sensitive adhesive to the platform 18, which when the patch is adhered to the skin can be released e.g. by pulling the platform 18 away from the skin 3. In the example of FIG. 15 as an alternative the platform 18 is tilted relative to the patch to “peel” the patch from the platform, as illustrated in FIG.15. In this FIG. 15, the patch 2 has a low-tack adhesive top surface which contacts the contact surface 182. To remove the patch 2, the base 10 can be tilted relative to the skin and the patch after the patch has been applied to the skin 2, such that the patch is peeled off the contact surface 182. However, depending on the type of adhesive, e.g. a linear movement may be suitable to separate the patch 2 from the platform 18.

In the example of FIG. 16, as another alternative the contact surface 182 and/or the patch 2 are provided with magnets. In this example, the patch 2 is provided with magnets 220 and the platform 18 is provided with a metal plate 181, in this example sunk under the contact surface 182 but alternatively the patch 2 may be provided with metal plates and the platform 18 with magnets for example. Also, the patch 2 and platform 18 may both be provided with one or more respective magnets, the magnets oriented with their magnetic field such that a magnetic attractive force is present between the patch and the platform. The magnet(s) may be a permanent magnet or an electromagnet, for instance. Also, instead of a metal plate another object magnetizable by the magnet may be embedded in the patch or, alternatively, in the platform 18. The magnetic force between the plate 181 and the patch 2 exceeds the gravitational forces but is lower than the adhesive force between the patch 2 and the skin 3. Thus, the patch 2 is held on the platform 18 but can be released once the patch 2 is applied on the skin 3 by simply moving the platform away from the skin 3 with a force exceeding the magnetic force.

The applicator may be provided with a mechanism to change the position of the magnet(s) relative to the magnetizable object (and/or the other magnets) in order to weaken or eliminate the adhesive force when the patch is to be released from the platform. The change may be relatively small, and for example a misalignment be induced by the mechanism that weakens or eliminates the adhesive force. The mechanism may engage directly on the platform and/or the patch, or, alternatively or additionally, the mechanism can cause a body to push the magnets away from the magnetizable object or the other magnets to weaken the adhesive force.

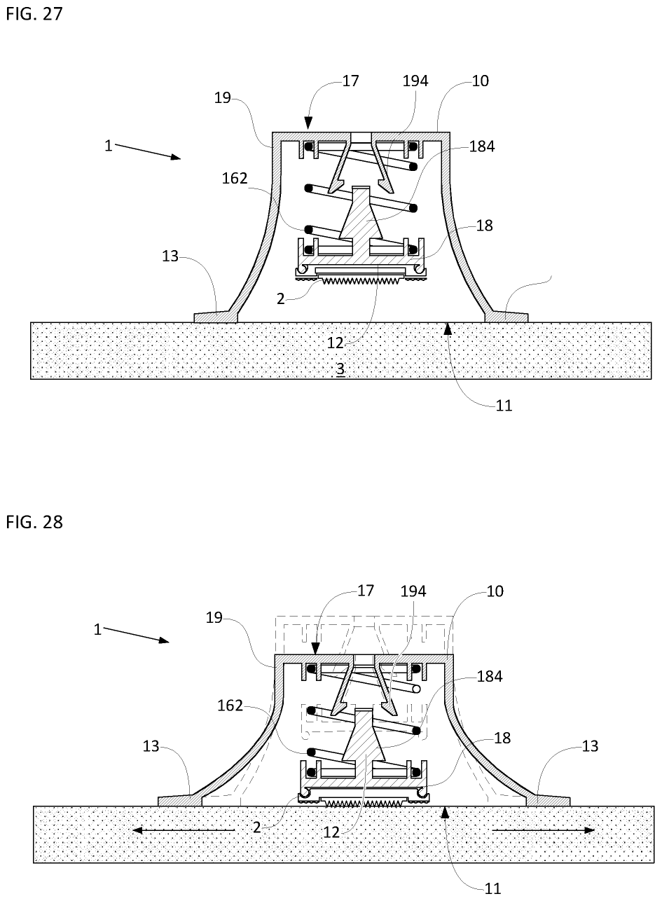

Likewise, as illustrated in FIG.27, as another alternative, the connector 122 may comprise a male part provided on the platform 18 which is, or can be, admitted in a female part provided on the patch 2. It will be apparent that the connector 122 may combine various connection types, such as those mentioned here.

The contact parts 13 may be implemented in any manner suitable for the specific implementation. In the example of FIGS. 1 and 2, multiple contact parts 13 are movable over the skin. As illustrated in FIG. 3, in which A shows the applicator 1 prior to stretching the skin 3 and B the applicator 1 after stretching the skin, the skin 3 can also be stretched with a single movable contact part 13 which can be moved relative to the skin and when moving moves the contacted skin relative to the base 10. In FIG. 3, another contact part 13′ holds the skin at another location in position relative to the base 10 in the direction of stretch. In the shown examples, the contact parts are all dedicated elements, but the patch held by the holder 12 may also be used as a contact part, together with the holder. Furthermore, in FIGS. 1 and 2, the contact parts 13 are separate elements, which seen in circumferential direction around the base 10 and parallel to the skin 3, are separated by respective gaps 133 between adjacent contact parts 13. Alternatively or additionally, some or all of the contact parts may be parts of the same, single element, such as of an elastically stretchable band or skirt enclosing the area of the skin 3, as in the example of FIG. 4 or be connected to each other at a position other than their proximal ends 132.

The contact parts 13 may be movable over the skin 3 in any manner suitable for the specific implementation. As shown in FIGS. 3 and 5, for example the contact parts 13 may be hingeably connected to the base 10, e.g. by means of a hinge 100. In the example of FIGS. 1-2, the contact parts 13 are each part of, or form, a respective flexing member arranged to flex under pressure exerted on the base 10 in the direction from the top 15 towards the skin side end 11, e.g. exerted by a hand, and a counterpressure from the skin 3 in the opposite direction, such that the contact parts 13 are moved away from each other in a direction parallel to the skin. The contact parts 13 can be of a resiliently deformable material, at least in the areas 134 where they flex. For instance, in FIGS. 1 and 2, the flexing area 134 is close to the proximal end 132, and as shown the contact parts 13 have a smaller cross-sectional thickness there than closer to the free-ends 131. Thus, under the pressure the contact parts 13 will mainly flex in the area of the proximal end 132. In an example, the contact parts 13 and the base-body 19 are of the same elastically deformable material. The contact parts 13 and base-body 19 and can for example be integrally moulded (e.g. by injection moulding, vacuum moulding or otherwise) or otherwise be monolithic. In such a case, for example, the base-body 19 can be made with thicker than the flexing areas 134 and hence be more rigid than the flexing areas 134 for example.

As mentioned, in the example of FIGS. 1-2, the contact parts 13 are separate parts, separated from each other. Each part has a fixed proximal end 132 connected to the base-body 19 and a distal, free-end 131 with a contact surface 130 for contacting the skin. The free-ends 131 are separated from each other by respective gaps 133, and movable relative to each other in at least a direction parallel to the skin 3. In this example, these separate parts comprise a number of legs, in this example equidistantly distributed in circumferential direction parallel to the skin-side end. As is best seen in FIG. 1, the proximal ends of the legs are attached to a common base, common to the legs, and each leg has a length between the proximal end and the free-end defined by the gap 133 between the legs.

As shown, seen in a direction from the top 15 to the skin-side end 11, the distal ends 131 project further than the proximal ends. Thus, a force in that direction on the base-body 19 will decompose in a component from the proximal end to the distal end and a component parallel to the skin 3. Accordingly, such a force can be used to transfer a stretching force onto the skin 3, and e.g. be used to flex or pivot the free-ends 131.

As can best be seen in FIG. 2, in this example the movable parts 13 comprise a contact surface 130 for engaging the skin to transfer the movement of the movable part onto the skin. More specific, in this example the contact surface 130 is a profiled skin contact surface with a friction enhancing profile. As shown, the profile can have a serrated cross-sectional shape, but it will be apparent that other shapes may be suitable as well. Additionally, or alternatively, the contact surface 130 may be of a friction enhancing material, such as a silicone-gel for example, as is illustrated in FIG. 15 with reference number 130′, for example. Also, for instance, the contact surface 130 may be coated with a friction enhancing coating.

The maximum amount the skin 3 can be stretched by the applicator 1 may be predetermined to not exceed a predetermined threshold, and preferably the threshold be below the amount of stretch which causes pain in the subject. More specific, the friction coefficient between the contact parts 13 and the skin 3 may be set such that when the stretch of the skin reaches the threshold, the force the skin 3 exerts on the parts 13 exceeds the frictional force. In such a case, the contact parts 13 will, instead of engaging with the skin 3 and stretching the skin 3, slide or slip over the skin 3 without noticeably stretching once the threshold is reached (or at least move with a significantly lower stretch per increase in distance between the contact parts 13.) This allows to have an upper limit on the stretch and thus avoid an amount of stretch that is uncomfortable to the subject.

In this example, the movable parts 13 are curved in axial direction of the base 10, i.e. from the top 15 to the skin-side end 11, and the contact surface diverges in the axial direction towards the skin-side end 11, such that the proximal ends 132 are more parallel to the axial direction and the free-ends are more parallel to the skin 3. This allows to render the friction coefficient less dependent, or independent from the normal force exerted on the top 15 in the direction of the skin 3. More specific, in case a higher normal force is exerted, instead of the frictional force increasing directly proportionally, a part of, or the complete, increase in the higher normal force will be absorbed by the movable parts 13 unrolling over the skin 3, and more specifically the part thereof contacting the skin, i.e. the contact surface 130 will become located further away from the free-ends, and closer to the proximal ends 132. Thus, the frictional force remains more or less constant, or at least increases less than the increase of the normal force. In addition, the orientation of the contact surface 130 relative to the normal force may change, and the angle between them increase, such that the force component parallel to the contact surface 130 becomes oriented more parallel to the skin 3. This allows to render the pressure exerted by the contact surface 130 on the skin 3 less dependent on the force exerted on the top 15.

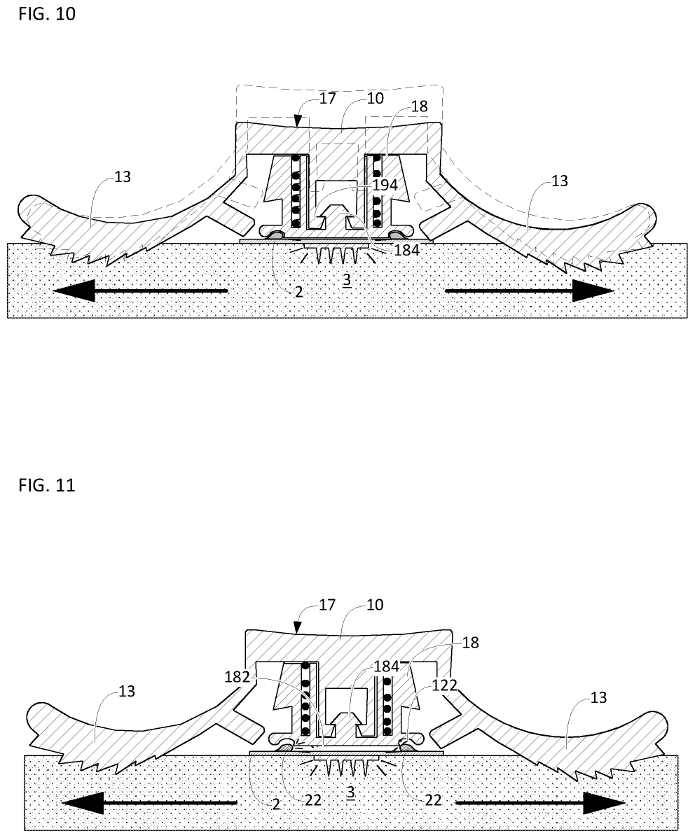

Additionally, due to the curved movable parts 13 the pressure perpendicular to the skin 3 is more smoothly transferred into a movement of the free-ends parallel to the skin 3. In this example, the skirt 103 seamlessly transitions into the movable parts 13 and both have a flaring shape. The skirt 103 is elastically deformable, at least in a radial direction perpendicular to the direction from the top 15 to the skin-side end 11. This provides a part of the flexing which allows to move the movable parts 13 over the skin 3. Additionally, or alternatively, the side walls of the space 102 may also be elastically deformable, at least in the radial direction, and thus provide a part of the flexing which allows to move the movable parts 13 over the skin 3 as well. More specific, in this example, as can be seen in FIG.7 and 9 for example, the elastically deformable skirt and/or side wall are stretchable in the radial direction, and are connected to the free-ends 131 to stretch when the free-ends 131 move away from each other. In this example, when the top 15 is moved towards the skin-side end 11, the pressure will cause the free-end 131 to move away from each other in the direction parallel to the skin 3, and the proximal ends 132 will move away from each other in that direction and stretch the elastically deformable parts between the proximal ends 132 and the top 15. At the same time, the proximal ends 132 move towards the skin-side end 11, in this example because the contact parts 13 will rotate around the proximal ends 132, thereby elastically bending the elastically deformable parts.

The actuator 16 may be implemented in any manner suitable for the specific implementation. In its simplest form, the actuator can be a hand of a human, e.g. of a medical practitioner or of the subject, and the interface 14 can be a suitably shaped grip or pressure contact surface that allows the human to exert the force required for the microneedles 21 to penetrate into the skin 3. However, other mechanical or electro-mechanical actuators are possible, such driven by a spring 161 as in the shown example or e.g. battery powered electro-mechanical actuators, just to name a few. Accordingly, the interface 14 may be any interface suitable to engage the applied type of actuator and to couple the actuator such that the movement of the microneedle can be actuated.

In this example, the applicator 1 comprises the actuator 16, and the actuator 16 is a, spring-based, mechanical actuator. The actuator 16 is cooperatively connected to the interface 14 for actuating a movement of the microneedle patch 2. More specific, the spring is arranged between the platform 18 and the base-body 19 to exert a force from the base-body 19 to the platform 18 in a direction towards the skin-side end 11. In this example the actuator 16 comprises a coil spring 161 which is compressed between the base-body 19 and the platform 18 in the direction from the top towards the skin-side end to store energy, and which by decompression can actuate the platform 18. However, the actuator may likewise be another type of actuator and be an external actuator, either a human or a machine powered actuator, which can engage with the interface 14.

In this example, the spring is compressed and thus an example of an actuator biased prior to use and thereby store energy which can be released to actuate the movement. More specific, in the example of FIGS. 1 and 2, the actuator 16 can during manufacturing have been biased, and the applicator 1 be provided in a biased state as illustrated.

The actuator may in case of an electrical actuator be triggered by a switch or other suitable control. In this example, the applicator comprises a contact surface 17 for a hand of the human operator and the actuator, as explained below in detail, is triggered by the manual pressure exerted on the contact surface 17. At the same time, the contact parts are coupled to the contact surface 17 by the flexing member connected at one side to the contact surface 17 and at another side to the contact parts 13. Thus, the flexing members will flex under the pressure exerted on the base by the hand and a counterpressure from the skin. Thus, the contact parts 13 will be moved away in a direction parallel to the skin and by the same gesture the actuator can be controlled.

As illustrated in FIGS. 6-14, when the actuator 16 is triggered, the actuator actuates the movement of the patch 2 towards the stretched skin 3, until the skin 3 is contacted and the microneedles 21 of the patch 2 penetrated the skin, as described above.

The actuator 16 can be arranged to control the movement of the patch 2 as well, e.g. in accordance with a pre-determined force profile of an accelerating force acting on the microneedle patch. The actuator 16 can be arranged to control the movement of the patch such that the platform exerts a maximum static pressure on the patch upon contacting the skin by the microneedle patch, of course sufficient to penetrate the skin. However, in this example, the actuator 16 is arranged to control the movement of the patch to have a velocity upon contacting the skin by the microneedle patch sufficient to penetrate the skin by impact. In the example of FIGS. 1 and 2 for example, the force exerted on the platform 18 is a function of the compression or extension of the spring 16, and the spring 16 has a natural length and an initial loaded state where the spring is compressed or extended relative to the natural length selected such that the platform 18 has a maximum speed when the patch 2 is at the skin-side end 11.

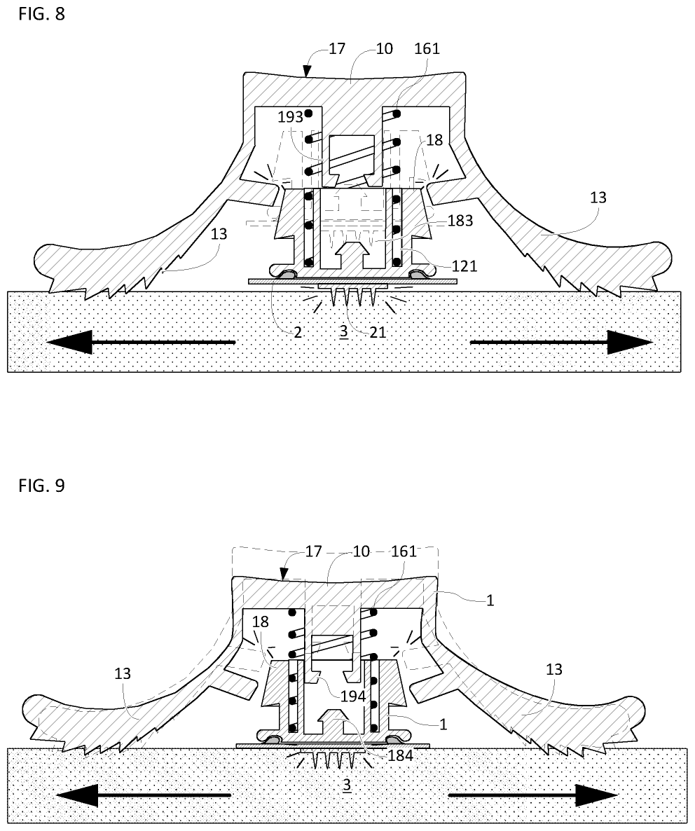

The holder 12 in this example further comprises a guide 192 for guiding the movement of the patch 2 along a predetermined path between the distant position and the skin contacting position. In this example, the path is a straight path, perpendicular to the skin 3, and the guide 192 comprises a straight protrusion 193 projecting in the space 101 of the base-body 19 with a longitudinal direction parallel to the path. The platform 18 is slidably mounted on the protrusion 193, and in this example comprises a hollow-sleeve 183 with an open end which is slid over the protrusion 193, and which as illustrated e.g. in FIGS. 8-10 defines at least a part of the path the platform 18 follows when moving relative to the base-body 19.

In this example, the protrusion 193 extends through the spring 161 and the hollow sleeve 183 is slidably mounted over the protrusion and the coil spring. More specifically, the hollows sleeve 183 is double walled, and the inner wall extends between the protrusion and the coil spring, whereas the coil spring extends in the interstitial space 12 1between the inner and outer wall of the sleeve 183. Thereby, not only is the movement guided but the spring 161 is hold in position. As shown, the skin-side end of the hollow-sleeve 183 is closed, and the spring 16 when compressed thus exert a force on that skin-side end, and hence push the platform relative to the base-body 19.

As explained in more detail with reference to FIGS. 6-14, the applicator 1 may further comprise a coupling between the contact parts 13 and the actuator 16. The coupling triggers actuation of the movement of the patch 2 when the contact parts 13 are moved a predetermined distance away from each other, and thus, in case the applicator is applied on the skin, when the skin has been stretched a predetermined amount by the contact parts 13. The coupling can be an electronic coupling, e.g. with a sensor which senses the distance and a trigger unit connected to the sensor which when the separation between the contact parts reaches the predetermined distance triggers actuation by the actuator. The coupling may also be an electro-mechanical coupling.

In the shown example, the coupling is a mechanical coupling. More precisely, the actuator 16 comprises a spring 161 arranged to be biased, and which engages with the holder 12 to exert on the holder, when biased, a force towards the skin-side end 11. However, the movement of the platform 18 is latched initially by releasable form closed connection between the contact parts 13 and the platform 18. The form-closed connection is releasable by a movement of the contact parts 13. In FIG. 2, this connection comprises a protrusion 180, which projects in a direction perpendicular to a direction of movement of the patch, and a recess 136 in which the protrusion is admitted. Although in this example the protrusion 180 is on the platform and the recess 136 in the contact parts 13, this may be the other way around for example. The protrusion is movable relative to the recess in the direction to leave the recess by a movement of the contact part. More specific, as illustrated in FIGS. 7 and 8, when the contact parts 13 are moved away from each other, the protrusion 180 is taken out of the recess 136 and the form-closed connection is released (as indicated with arrows A in FIG. 7) when the free-ends 131 of the contact parts 13 are a certain distance from each other.

Thus, the contact parts 13 form a control which engages on the releasable latch to control the state of the releasable latch. This control ensures that the latch enters into the release state when the contact parts are moved the predetermined distance away from each other.

In the shown example, coupling also couples the movement of the contact parts 13 to the contact surface 17. More specific, when a pressure is exerted on the contact surface 17 by a hand of an operator or otherwise, the pressure is transferred on the contact parts 13 via the base-body 19, and the contact parts 13 will move. At the same time, the contact parts 13 latch the platform 18, and by the movement of the contact parts 13 the latch is unlatched.

In the shown examples, the patch 2 is movable relative to the base 10, and more precisely the platform 18 on which the patch 2 is mounted is movable relative to the base-body 19. The coupling in such a case be arranged to trigger actuation of the movement of the patch relative to the base when the contact part is moved the predetermined distance away from the other contact part. In the example of FIGS. 1-2, when the contact parts are separated corresponding to the predetermined distance, the protrusion 180 is outside the recess 136 and thus the form-closed latching connection is unlatched. As a consequence, the platform 18 can, and will be, moved by the spring actuator 16 relative to the base-body 19.

In the example of FIG. 1-2, the coupling is arranged to trigger actuation of the movement of the patch when the patch is at a distance from the skin. However, it will be apparent that e.g. as in FIGS. 22-24 alternatively, the patch may contact the skin already when the movement is triggered.

In this example, the applicator 1 further comprises a latch which latches movement of the holder 12 in a direction away from the base at a predetermined point after movement has started, such as upon or after penetration of the skin 2. More specific, the latch comprises a snap-fit connector 184, 194 between the protrusion 193 and the hollow sleeve 183. As illustrated in FIG. 2, the snap-fit connector is in an initial state unconnected and arranged to connect after the movement of the patch 2 has started, by moving the base-body 19 towards the platform 18 and thereby establishing the snap-fit connection, as illustrated in FIG. 11. This allows to then take the platform 18 away from the skin 3 by simply moving the base-body 19. As a consequence, the patch 2 will release from the platform 18 because the adhesive force between the patch 2 and the skin 3 exceeds the force attaching the patch 2 to the platform 18, and the snap-fit connection is stronger than this attaching force.

Referring now to FIGS. 6-14, the applicator 1 may perform a method of applying a patch to the skin as follows. In those FIGS., the previous state of the applicator 1 is indicated with dashed-lines.

As shown in FIG. 6, initially the applicator 1 is placed, with the patch 2 mounted in the holder, with the skin-side end 11 on the skin 3. For example, the applicator may be placed by a medical practitioner or be used to self-administer by a subject. In this respect, the subject can be a human or an animal. The applicator may be placed on a part of the body of the subject selected from the group: head, ear, neck, limb, arm, upper arm, lower arm, hand, leg, upper leg, lower leg, foot, torso, chest, abdomen, pelvic region, back, shoulders, buttocks. For example, applicator may be placed to the inside of the lower arm. Thereby, a relatively low amount of force is needed to penetrate the skin, since the skin is relatively thin in that area, and additionally few preparations are required pre- and post application of the patch because this body part has not that much hair. The applicator may be adapted to the thickness of the skin of the selected body part, and e.g. to exert more force on the microneedle patch if the applicator is for a part with relatively thick skin layers, such as at a buttock, compared to the force of an applicator for a part with relatively thin skin layers, such as an ear. It will be apparent that, e.g. in case of self-administration, the body part is preferably within reach of the hands of the subject.

Depending on the specific implementation, the process of applying the patch after the applicator has been placed on the skin may comprise one or more phases, such as: a stretching phase in which the skin is stretched, a non-contact phase at the beginning of which the patch 2 is at a distance from the skin 3 and at the end of which the patch contacts the skin, a non-penetrating phase at the beginning of which the patch contacts the skin 3 but the microneedle does not noticeably penetrate the skin, a penetrated phase at the beginning of which the patch contacts the skin 3 and the microneedle penetrates the skin, and a separation phase at the end of which patch contacts the skin 3, the microneedle penetrates the skin 3 and the applicator 1 is separated from the patch 2.

The phases may be performed in the listed order, e.g. succeed each other or overlap, depending on the specific implementation. When using the example of FIGS. 1 and 2, as more clearly apparent from FIGS. 6-12, for example, the process does strictly speaking comprise all of those phases, and they succeed each other in the order listed above, but the non-penetrating hpase is very short and the applicator 1 transitions almost instantaneously from the non-contact phase to the penetrated phase. When using the example of FIG. 19-20, for instance, the non-contact phase coincides process with the stretching process and once the skin 3 is stretched the patch 2 contacts the skin 3 without penetration. When using the example of FIG. 27, for example, the stretching phase overlaps with both the non-contact phase, the non-penetrating phase, and the penetrating phase and like the example of FIGs.1 and 2, the applicator 1 transitions almost instantaneously from the non-contact phase to the penetrated phase.

The applicator 1 is placed on the skin 3 with the contact parts 13 contacting with their respective contact surfaces 130 the skin 3. The spring-actuator 16 is biased, and exerts a force on the platform 18, but the movement of the platform 18 relative to the base-body 19 is latched, in this example by the form closed connection.

A pressure towards the skin 3 may then be exerted on the surface 17, e.g. manually. Due to the counter pressure of the skin 3, the free-ends 131 of the contact parts 13 will tend to move away from each other in a direction parallel to the skin 3, and thus start stretching the skin 3 due to the friction between the skin 3 and the contact surfaces 130. As illustrated in FIG. 7, the contact parts 13 will then move, more precisely the free-end 131 will move over the skin 3, and the contact surface 130 will stretch the skin. At the same time, the base-body 19 moves towards the skin and the coupling will trigger the spring-actuator 16 when the free-ends are moved a predetermined distance away from each other. More specific, as illustrated with arrows A in FIG. 7, the latch (formed in this example by the protrusion 180 and recess 136) will be unlatched because the movement of the leg-shaped contact parts 13 takes the protrusion out of the recess 136. As shown in FIG. 8, when the latch is unlatched, the actuator is triggered by the coupling, and the actuator will move the platform 18 relative to the base-body 19 towards the skin 3. The platform 18 is thus moved outside the space 101 in which it was held, until the patch 2 contacts the skin 3, and in this example the microneedles 21 penetrate the skin 3 upon contact with the skin 3 due to the mass of the platform and the velocity thereof. As mentioned earlier, the microneedles can penetrate in at least into, or at least through, the stratum corneum and optionally deeper as described above.

As illustrated, the spring-actuator is coupled to the platform, in this example via the interstitial space 121 between the walls of sleeve 183 and abuts to the closed end of the interstitial space 121. Thus, once the coupling unlatches the platform 18 from the contact parts 13 (which are connected to the base-body 19), the movement of the platform 18 relative to the base-body 19 is triggered thereby and the actuator 16 actuates the movement towards the skin 3. The movement is guided, in this example by the protrusion 193 and sleeve 183 but other types of guides may additionally or alternatively be used as well.

As illustrated in FIG. 9, the patch 2 is applied to the skin 3 by the movement driven by the actuator 16. To release the patch 2 from the platform 18, the platform 18 may then be moved away from the skin 3. In this example, to that end, the base-body 19 is moved towards the skin 3, such that the base-body part 194 of the snap-fit connector comes to engage with the platform part 184 of the snap-fit connector and the snap-fit connection is established. In FIG. 9, the protrusion 193 with the base-body part 194 of the snap-fit connector on its distal end, slides in the sleeve 183 towards the skin 3 until the base-body part 194 snaps the platform part 184. As shown in FIG. 10, the snap-fit connection is thereby established and the platform 18 can be separated from the patch 2 by moving the base-body away from the skin 3, as illustrated in FIG. 11 which releases the attachment of the patch 2 from the contact surface 182 of the platform 18. The applicator 1 without the patch 2 may them be completely removed from the skin 3, and e.g. be disposed of (in case of a disposable, single use applicator) or be re-used with a new patch 2.

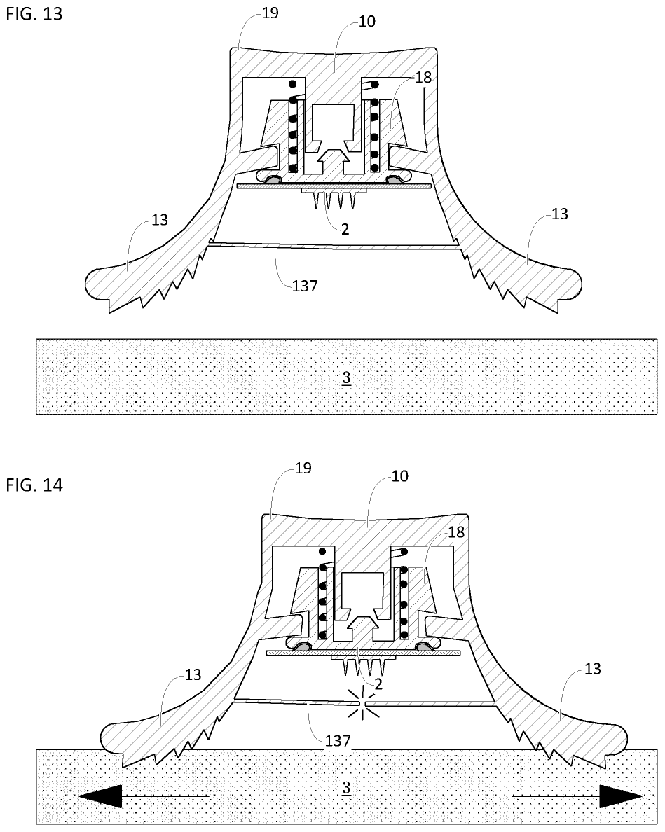

As illustrated in FIGS. 13 and 14, the applicator can comprise a releasable bond 137 between the contact parts 13 which inhibits movement of the contact parts 13. The releasable bond can be releasable upon exerting a predetermined amount of force in a direction of movement of the contact parts. This allows to determine whether or not the applicator has been used already, and avoid re-use of a disposable applicator for example. As in the shown example, the releasable bond can be destructively releasable. For instance, upon movement of the contact parts 13 a predetermined amount, and in this example prior to the patch 2 perforating the skin, the bond 137 may break. The releasable bond can e.g. comprise a seal between the contact parts which opposes moving the contact parts towards each other. In this example, the releasable bond is a seal which separates an inside of the applicator 1 in which the patch 2 is provided from the outside. The seal, until being opened, seals off the inside and thus safeguards that the patch 2 remains sterile. More specific, in this example, the seal comprises a membrane which, when the contact parts 13 are moved sufficiently away from each other, is torn apart. To that end, for example a pattern of local weakenings may be provided in the membrane to ensure the membrane tears in a predetermined pattern, e.g. to not obstruct the path of the patch 2 from its initial position to the position thereof on the skin 3. The membrane may for example be made of a wrinkling material, which wrinkles when the membrane is torn apart, such that the membrane moves itself to the sides of the inside of the applicator 1, defined by the contact parts 13 and the base 10.

In case the patch 2 is pre-mounted, the seal obviates the need for a separate package to maintain the patch 2 sterile, and the tearing apart allows to reduce the number of operations required by the operator to handle the applicator 1.

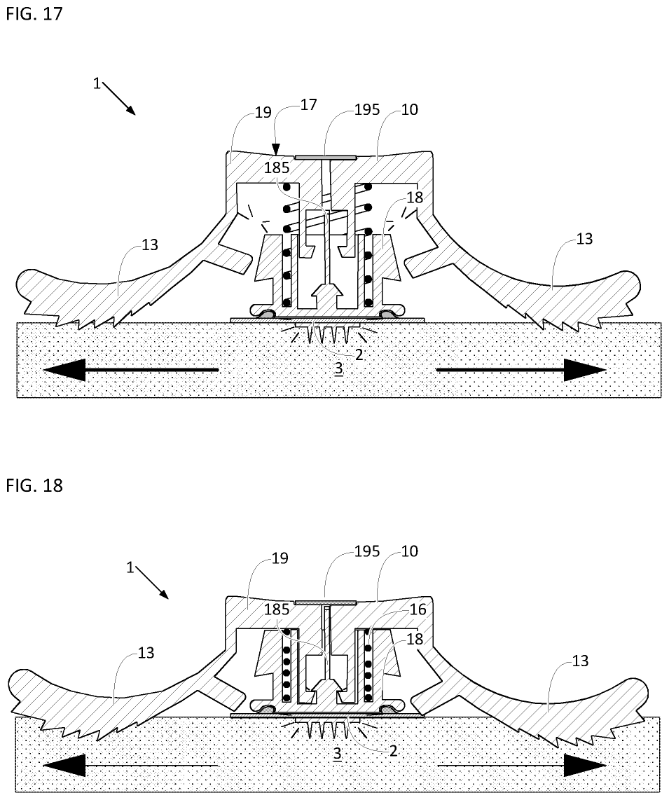

As illustrated in FIGS. 17 and 18, the platform 18 and the base-body 19 may be provided with a penetration sensor 185,195 which provides to the operator of the applicator 1 a tactile, visual or other for humans perceptible feedback when the patch 2 has been applied to the skin 3 and/or when the pressure applied to the patch 2 has reached a threshold value. The shown example is a tactile sensor, which comprises an upright element 185, in this example pole-shaped, which extends from the platform 18 towards or through a passage in the base-body 19 and which is provided at the top with a visible marker. The sensor further comprises a transparent plate 195 at the contact surface which covers the passage. As shown, initially the upright end of the element 185 is below the top 15 with the pressure surface 17. When the platform 18 with the patch 2 contacts the skin 3, the platform 18 does not move further towards the skin 3, but the base-body 19 will move towards the skin 3 and thus towards the platform 18 while at the same time in this example the amount of pressure exerted on the platform 18 will increase (because the spring 161 is compressed and the actuator). As a consequence, the upright end of the upright element 185 will at a certain distance come to abut to the plate 195, and the marker be visible through the plate 195. This thus provides a, for human beings perceptible, signal that the user can stop pressing. The penetration pressure sensor preferably provides this signal when the pressure exerted by the actuator 16 on the platform 18 exceeds a predetermined threshold. In this example for instance, the pressure is exerted by the spring and the length of the upright element 185 is such that when the spring is compressed to a predetermined amount (and hence the force and pressure to a corresponding level), the end of the upright element 185 will become visible through the plate 195.

In this example, the marker becomes visible at the same time the snap-fit connection interlocks the platform 18 and the base-body 19, and thus serves as a sensor for this as well. However, alternatively or additionally, other feedback is possible as well. For example, the snap-fit connection 184,194 may provide a tactile or auditive feedback when the platform 18 and the base-body 19 interlock and thus indicate that the applicator 1 may be removed from the skin 3.

Referring now to FIGS. 19-26, the example shown therein uses the same basic concepts as the example of FIGS. 1 and 2 but differs in the following.

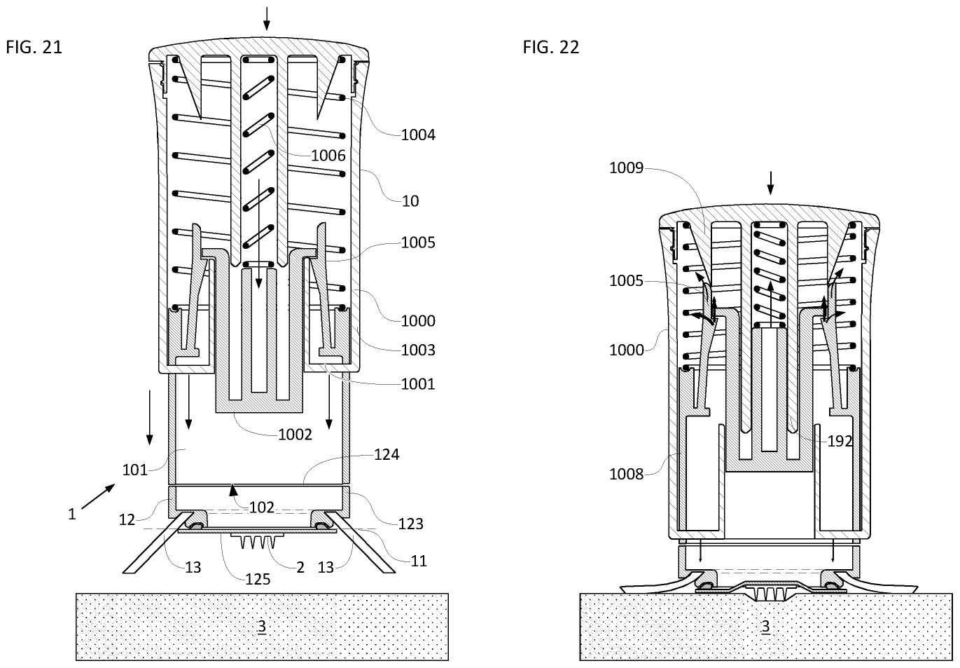

In this example, the base 10 does not comprise a body 19 with a holder 12 held at a distance from the skin-side end 11. Rather, as best seen in FIG. 21, in this example the holder 12 is at or close by the skin side end 11 and the base 10 comprises a housing 1000 with a bore 1001 extending towards the holder 12, and a mass 1002 provided in the bore 1001. The mass 1002 is movable towards the holder 12 to generate an impact-force on the holder 12 which causes the holder 12 to move towards the skin, and a patch 2 mounted on the skin-side end 125 to penetrate the skin 3. The base 10 further comprises a kinetic transducer 1003 for converting a movement of the housing relative to the holder 12 into potential energy of the mass 1003, and a potential energy storage 1004 for storing the potential energy. In this example, the storage 1004 is a spring, and the transducer 1003 formed by a compression contact which compresses the spring 1004 when the housing 1000 is moved towards the holder 12. The base 10 further comprises a release 1005 for releasing the potential energy; and a kinetic transducer 1006 for converting the stored potential energy into kinetic energy of the mass upon release.

The holder 12 also differs from the example of FIGS. 1 and 2. In the example of FIGS. 19-26, the base 10 comprises a space 101 at the skin-side end which has an opening 102 facing the skin-side end 11. The holder 12 is formed by an insert 123 and the insert 123 can pass, at least partially through the opening 102 to be connected to the housing 1000. In this example, the insert 123 then closes of the opening 102. The insert piece 123 has a proximal side 124 to be admitted into the space 101 and to be interlocked with the base 10, and an exposed, skin-contacting side 125 provided with the contact parts 13 for contacting the skin. As shown, the contact parts 13 project towards the skin 3, and the patch 2 is, seen in a direction from the top of the housing 1000 to the skin 3 is located between the skin-side 125 and the free-ends of the contact parts 13.

Furthermore, the contact parts 13 are formed by blocks 1007 of a resilient material shaped to contact the skin and deform under a shear stress induced by pushing the base 10 onto the skin 3, the blocks are placed around the opening 102.

In this example, the blocks 1007 are part of the insert 123. Thus, the contact parts 13 can be selected by selection of a specific insert 123. This allows to select, e.g. by the patient himself or by a medical practitioner, a contact part 13 with characteristics, such as length, with and resilience, suitable to stretch the skin in the manner deemed desired for the specific treatment and/or the skin at the place of application of the patch, for example based on location of application, age, skin type or other characteristics of the subject. E.g. out of a set of inserts 123 which vary at least in these characteristics, a contact part 13 with the suitable characteristics may be taken and mounted on the applicator 1. Such selectable contact parts 13 may be provided in another manner and be applied to another applicator than shown in FIG. 19-20. For example, the contact part 13 could be a separate part, separate from the platform, and be exchangeable without changing the platform. and, releasably attachable to another part of the application

The example of FIGS. 19-20 may operate as illustrated in FIGS. 21-26. As shown in FIG. 21, prior to being placed on the skin 3, the contact parts 13 are undeformed, and the patch 2 is provided on the skin-side 125 of the insert 123. The applicator 1 may then be placed on the skin 3, and a pressure be exerted, as indicated with the arrow from the top towards the skin 3. As a consequence, and due to the counterpressure of the skin 3, the contact parts will flex such that the free-ends thereof are at the same level as the patch 2, and the patch 2 comes to contact the skin 3. However, here the patch 2 does not penetrate the skin, or only partially, but the microneedles may depress the skin in the area of penetration, as shown in FIG. 22. It will be apparent though that the microneedles may partially penetrate the skin and e.g. make shallow perforations of a depth significantly less than the final depth.

Due to the pressure and counterpressure, the housing 1000 is moved towards the insert 123, and as shown the springs 1004,1006 compressed. As shown, here the spring 1004 is placed inside the housing and compressed between a top end of a lower body 1003 slidably mounted in the space 101, the lower body 1003 thus forming the transducer, and the, closed, top of the space 101 formed by an upper body provided with the bore 1001 in which the mass 1002 is mounted.

The lower body 1003 is in turn interlocked with the insert 123. The mass 1002, and lower body 1003 do not move relative to the insert 123 when the upper body is moved towards the insert 123. As shown, between the top of the space 101 and the mass 1002 another spring 1006 is present which forms the kinetic transducer, and which is compressed as well by the movement of the upper body.

As shown in FIG. 22, the springs 1004,1006 are thus compressed and the kinetic energy of the movement of the upper part of the body is converted into potential energy of the springs 1004,1006. Movement of the mass 1002 is prevented though by a stop 1005, formed in this example by a blade-shaped projection of the lower body 1003 toward the top of the upper body which has a stepped shape 1011 over which a projecting edge 1012 of the mass 1003 projects and which is positioned between the edge and the insert 123 such that the mass 103 cannot move towards the insert 123. As shown with the arrows in FIG. 22, the space 101 is provided with projections 1009 with a surface which is inclined relative to a direction of movement of the upper part of the housing, and over which the stop can slide when the upper part is moved further towards the insert 123. Due to the inclined surface, the stepped shape 1011 is pushed away from the projection 1012 in a direction perpendicular to the direction of movement. The stepped shape 1011 is pushed so much that it does not overlap in the direction of movement with the projection 1012, and the mass 1003 can thus move towards the insert 124 and the patch 2. Naturally, the inclined surface may be located on the transducer and the blade-shaped projections on the moving mass 1002.

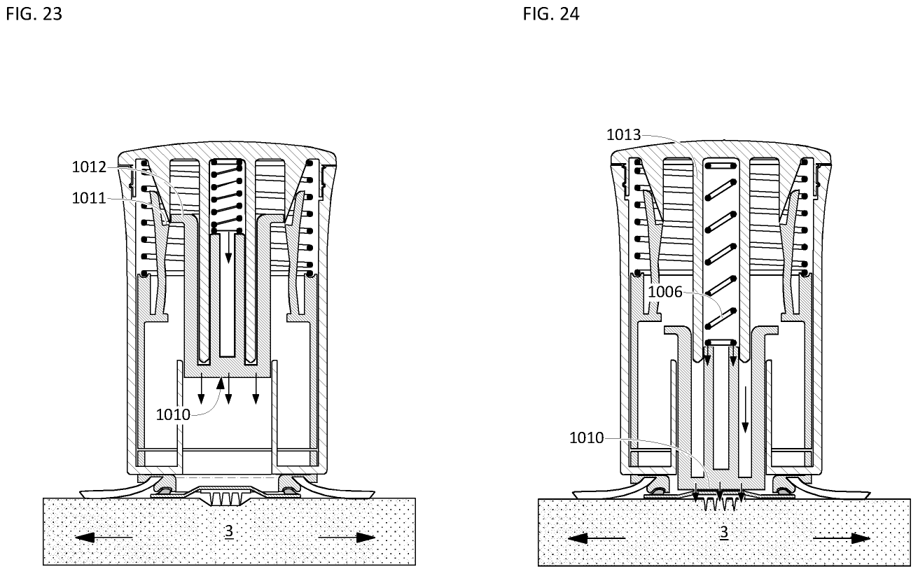

As illustrated in FIG. 23, the force the, compressed, spring 1006 has been exerting on the mass 1003 thus actuates the movement of the mass 1003 towards the insert 123, as indicated with the arrows. The spring 1006 will thus decompress and convert its potential energy into kinetic energy of the mass 1003, and thereby act as kinetic transducer. As illustrated in FIG. 24, the mass 1003 will impact on the insert 1003, and more specifically on the patch 2 and as a consequence the microneedles will perforate the skin 3.

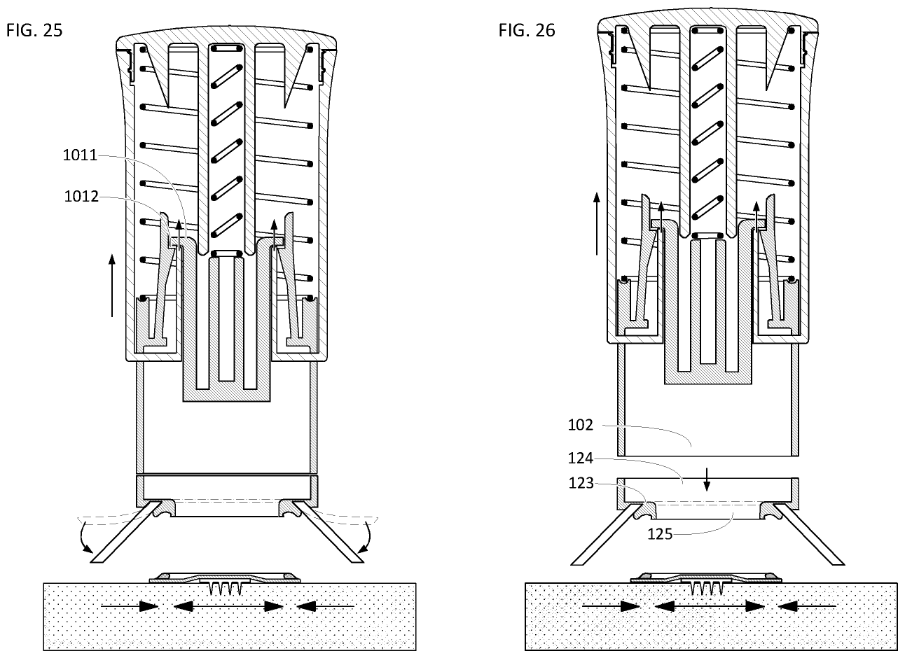

The applicator 1 may then be removed. As illustrated in FIG. 25, the edge of the opening in the bore 1001 may for example overlap in the direction of movement with the projections 1011, such that when the upper part of the housing is moved away from the skin 3, this edge comes to abut to the bottom of the projection 1011 and moves the mass 1003 upwards relative to the bottom part 1003. The projection 1011 of the mass 1003 then slides over the stepped edge 1012 and is thus again held by the housing 1000 and at a distance from the insert 123. By moving the upper part, the bottom part 1003 and the insert 123 may be moved away from the skin 3. As shown in FIG. 25 with the dashed lines, the contact parts 13 then resume their neutral position and relax.

As illustrated in FIG. 26, the insert 123 may then be separated from the housing 1000 and e.g. be thrown away.

Referring now to FIGS. 27-29, the example shown therein uses the same basic concepts as the example of FIGS. 1 and 2 but differs in the following.

As shown, in this example the actuator is not part of the applicator 1. The interface for the actuator is in this example formed by the top of the applicator base 10, and more specifically as a contact surface 17 on which e.g. the subject or another person, like a medical practitioner, can exert pressure. For example, the contact surface 17 may as shown be shaped such that a hand can press on the surface 17 and manually push the top of the applicator base 10 towards the skin 3 but other type of actuators coupled to the interface may alternatively be used.

Furthermore, in FIGS. 27-29, the actuator engages on the base-body 19, and the interface is provided on the base-body, to actuate a movement of both the base-body 19 and the platform 18 towards the skin 3. Although in this example the platform 18 does not move relative to the base-body 19, it will be apparent that the base-body 19 and the platform 18 may have different speeds when moving to the skin 3.

The shown example further comprises a pressing unit 162 which exerts on the platform 18 a pressure when the patch 2 contacts the skin which is transferred by the platform 18 to the patch 2 to penetrate the skin 3. The pressure exerted by the pressing unit 162 is coupled to the movement of the contact parts 13. Thus, the point in time the skin 3 is penetrated is coupled to the amount of stretch of the skin 3. Thus, the amount of pressure needed can be well controlled.

Another difference is that the movable platform 18 is not latched to the base-body 19 prior to triggering actuation. Instead, the movable platform 18 is movably attached to the base-body 19. In this example a compression spring 162 which has its axis of compression parallel to the direction extends between the platform 18 and the base-body 19, and which is compressible by a movement of the platform 18 towards the base-body 19.

The movement of the platform 18 towards to the skin-side end 11 is coupled to the movement of the base-body. More specific in this example, the path of the platform 18 from an initial position to a skin-contacting position is unrestricted and when travelling along the patch 18, the force towards the skin 3 exerted on the platform 18 by the movement of the base-body 19 (in this example via the spring 162) is larger than forces exerted by e.g. the skin 3 or other external elements in the opposite direct (in this example these opposite forces are zero). Thus, the spring 162 is not compressed, or at least not more than in the initial position.

The coupling of the movement of the platform 18 and the base-body 19 is interruptible, and in this example is automatically interrupted at a predetermined point, in this example when the patch 2 touches the skin 3. After the predetermined point, the base-body 19 is moved towards the skin 3 and towards the platform 18 until the base-body 19 and the platform are at a predetermined distance from each other. When at the predetermined distance, the platform 18 and the base-body 19 are interlocked, in this example by a snap-fit connection 184,194, at least in a direction away from the skin 3. Thus, from that point on, the movements of the platform 18 and the base-body 19 are again coupled and more specific in this example the platform 18 is no longer movable relative to the base-body 19. This allows, as shown, to move the platform 18 relative to the patch 2 applied to the skin 3, and separate the patch 2 from the applicator 1.

The example of FIGS. 27-29 operates as follows. Initially, the applicator 1 is placed on the skin 3, with the contact parts 13 contacting the skin 3. As shown, the platform 18 and the patch 2 are at a distance from, and do not contact, the skin 3. This allows (by the way like in the examples of FIGS. 1-5) to move the applicator 1 over the skin and reposition the applicator without the patch 2 being contaminated or becoming unsterile. As shown, the base-body 19 may then be moved towards the skin-side end 11 by the actuator engaging on the interface, in this example on the contact surface 17 at the top of the base-body 19. Due to the coupling between the movement of the base-body 19 and the contact parts 13, the contact parts 13 move away from each other and the skin 3 is stretched, as illustrated with the arrows in FIG. 28.

Parallel in time, the platform 18 is moved towards the skin-side end 11, because coupled to the movement of the base-body 19 until the patch 2 contacts the skin 3, as shown in FIG. 28. As shown, the platform 18 and the base-body 19 are initially at an initial distance from each other, and this distance remains the same during this pre-contact phase. However, alternatively, during the pre-contact phase, the distance may vary while both the distance between the platform 18 and the skin-side end 11 and the distance between the base-body 19 and the skin-side end 11 reduce.

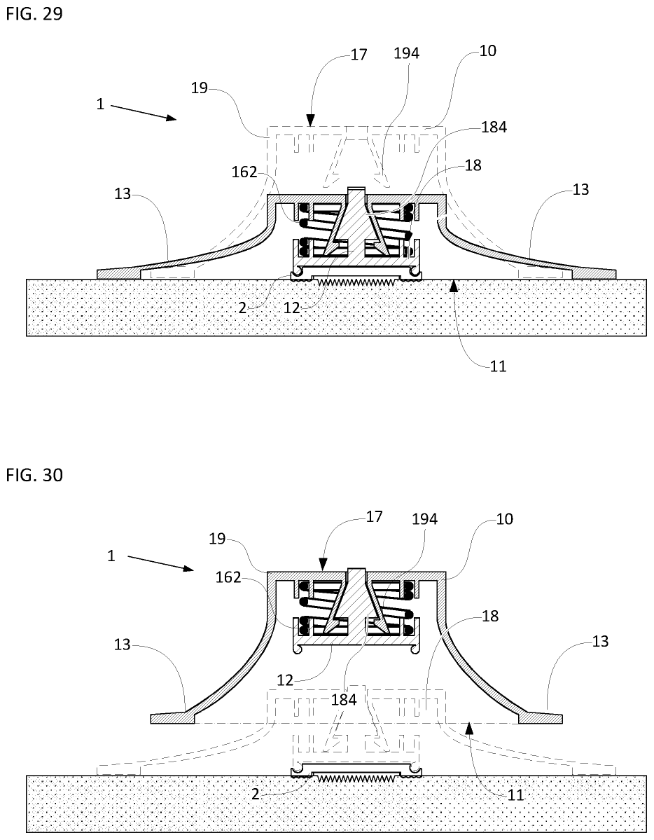

When the patch 2 contacts the skin 3, further movement of the platform 18 into that direction is inhibited by the skin 3. As mentioned, the coupling with the movement of the base-body 19 is interrupted. When, as illustrated in FIG. 29, the base-body 19 is moved further towards the skin 3, the base-body 19 is moved towards the platform 18, and the distance becomes less than the initial distance between them. A skin-wards pressure is exerted on the platform 18 which causes penetration of the skin by the microneedles in the patch 2. More specific, the movement causes a compression of the spring 162 (because the body side thereof is moved towards the other, platform side end which remains in position relative to the skin 3). This compression causes the spring 162 to exert on the platform 18 a force towards the skin 3 which is transferred onto the patch 2 to push into the skin 3. As show, at a predetermined point, when the platform 18 and the base-body 19 are moved towards each other in this penetration phase, the distance between them becomes an interlocking distance, in this example smaller than their initial distance, which triggers the interlocking by means of the snap-fit connection. As illustrated in FIG. 29, the base-body 19 and the platform 18 are then interlocked, and the movement of the platform 18 in the direction away from the skin 3 is then coupled to the movement in that direction of the base-body.

As show in FIG. 30, the base-body 19 may then be moved away from the skin 3. As illustrated, the patch 2 has been applied to the skin 3 and the adhesive forces between the patch 2 and the skin 3 are stronger than the holding forces that hold the patch 2 onto the platform 18, while on the other hand the coupling between the platform 18 and the base-body 19 is stronger than those. Thus, due to the interlocking, the movement of the base-body 19 causes a movement of the platform 18 which leads to the patch 2 being release from the platform 18 and remaining at the skin 3. As shown, in this separation phase the base-body 19 is removed from the skin, and the distance between the skin-side end 11 and the patch 2 increases. At the same time, the distance between the base-body 19 and the platform 18 remains at the interlocking distance, although this may reduce or even increase as long as the increase remains less than the distance between the skin-side end 11 and the patch 2.

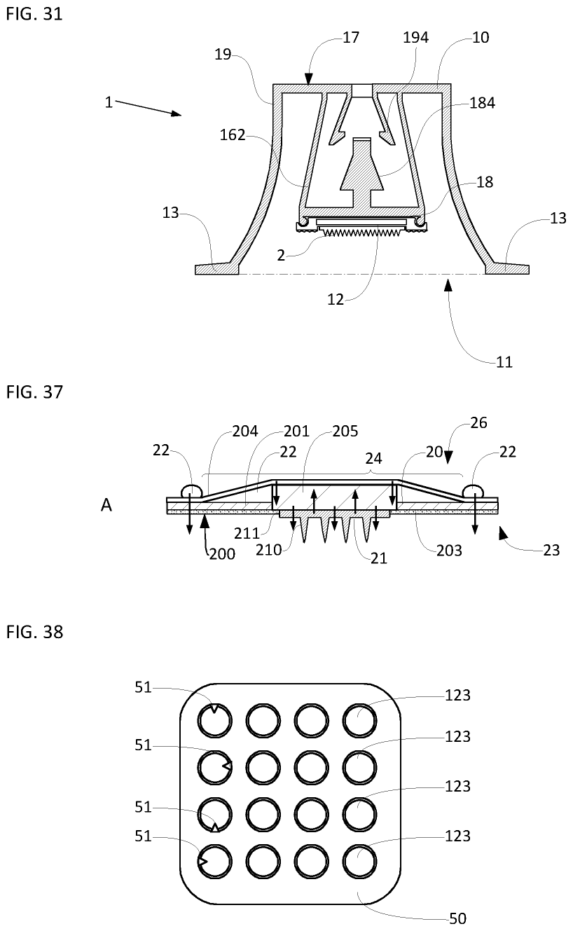

FIG. 31 shows a modification of the example of FIG. 27. As shown therein, the connection between the base-body 19 and the platform 18 may be implemented in another manner as well. For instance, in this example the spring 162 is not a coil-spring but a blade spring arrangement of several blades, which with one end are attached to the base-body 19 and with the other end to the platform 18. In the penetration phase, the blades flex due to the reduced distance between base-body 19 and the platform 18, and accordingly a pressure towards the skin 3 is exerted on the platform 18 by the flexed blades (provided of course that the flexed blades are prevented from relaxation into their initial state, e.g. by the base-body 19 being blocked from moving by the counter pressure caused by the flexed blades thereon, such as be a hand pressing on the contact surface 17). It will be apparent that other connections may likewise be implemented.

In FIG. 31, the spring is an integral part of the base 10 and connects the holder 12 with the platform 18 to the base-body 19. Thus, the base-body and the spring, and optionally the holder 12 are monolithically made, and thus manufacturing of the applicator 1 can be simplified. The spring 162 can e.g. be integrally moulded together with the platform 18 to the base-body 19, e.g. in a single injection moulding shot, or the application can e.g. have been 3D-printed by additive and/or subtractive manufacturing for example. It will be apparent that other types of flexible connections, such as a coil spring may likewise be implemented as integral part of the base 10.

As best seen in FIG. 29, in this example the platform 18 and the base-body 19 are provided with a penetration pressure sensor as well which provides to the operator of the applicator 1 a tactile feedback when the patch 2 has been applied to the skin 3. The shown example is a tactile sensor, which comprises an upright element 185 which extends from the platform side part 184 of the snap-fit connection and a passage in the base-body 19 facing the upright element 185. As shown, initially the upright end of the element 185 is below the top 15 with the pressure surface 17. When the platform 18 with the patch 2 contacts the skin 3, the platform 18 does not move further towards the skin 3, but the base-body 19 will move towards the skin 3 and thus towards the platform 18. As a consequence, the upright end of the upright element 185 will at a certain distance come to pass through the passage and come to projection out of the contact surface 17. This will be felt by the user of the applicator 1 and thus provides a, for human beings perceptible, signal that the user can stop pressing. The penetration pressure sensor preferably provides this signal when the pressure exerted by the actuator 16 on the platform 18 exceeds a predetermined threshold. In this example for instance, the pressure is exerted by the spring 162 on the platform 18 and the length of the upright element 185 is such that when the spring 162 is compressed to a predetermined amount (and hence the force and pressure increased to a corresponding level), the end of the upright element 185 will come to project and the tactile feedback signal provided.

### Patch

Referring to FIGS. 32-33, a microneedle patch 2 is shown therein. The shown example may be used in an applicator 1 as described above, but it will be apparent that the applicator can use other patches and, vice versa the patches may be applied in another manner on the skin of a subject.

The microneedle patch may be applied by a medical practitioner or be used to self-administer by a subject. In this respect, the subject can be a human or an animal. The microneedle patch may e.g. be applied on a part of the body of the subject selected from the group: head, ear, neck, limb, arm, upper arm, lower arm, hand, leg, upper leg, lower leg, foot, torso, chest, abdomen, pelvic region, back, shoulders, buttocks. For example, the patch may be applied to the inside of the lower arm. Thereby, a relatively low amount of force is needed to penetrate the skin, since the skin is relatively thin in that area, and additionally few preparations are required because this body part has not that much hair. The applicator may be adapted to the thickness of the skin of the selected body part, and e.g. to exert more force on the microneedle patch if the applicator is for a part with relatively thick skin layers, such as at a buttock, compared to the force of an applicator for a part with relatively thin skin layers, such as an ear. It will be apparent that, e.g. in case of self-administration, the body part is preferably within reach of the hands of the subject.

The shown example of microneedle patch 2 comprises a flexible sheet 20 with at a skin-contacting side 23 a skin-adhesive surface 200 for attaching the patch 2 to the skin 3 of a subject. The patch 2 further has an upper surface facing 26 away from the skin-adhesive surface 200. The upper surface 26 is separated from the skin-adhesive surface by at least the flexible sheet 20. Although the flexible sheet 20 may be implemented in any other manner suitable for the specific implementation, and e.g. be a single layer sheet, the shown example comprises at least in the region a laminate of multiple layers 201-203 adhered to each other.

The patch 2 may be used to perform operations between the microneedles and the body of the subject, e.g. administer a pharmaceutically active substance to the subject, or collect, through the microneedles, a substance from the subject, e.g. dermally or transdermally, sense properties of the body or modify the body (e.g. by heating of, or sending electrical current into, the perforated area of the skin). To that end, the patch 2 can be placed on the skin of a subject with the skin-adhesive surface contacting the skin, e.g. with an applicator as described above. The skin may have been stretched before placing the patch or be stretched during placement. The microneedles of the patch may upon, or after, contacting the skin penetrate at least the epidermis of the skin. For example, the microneedles may perforate the stratum corneum without piercing through, or pierce through the stratum corneum, and any intermediate layers, until into one of the following skin layers (without piercing that layer): stratum lucidum, stratum granulosum, stratum spinonsum, stratum basale, basement membrane, papillary dermis, reticular dermis.

In this example, one of the layers is an intermediate or base layer 201 and the layers 201-203 further comprise an adhesive layer 203 at the skin-side of the base layer 201. The skin-adhesive surface 200 is formed by the exposed surface of the adhesive layer 203 which covers the base layer 201 in the areas where the patch is to be attached to the skin. In this example, for instance, the skin-adhesive surface 200 encloses a microneedle region 24 delimited by a stiffening body 22 as will be explained below. Outside the region 24, the skin-adhesive surface 200 covers the entire skin-side of the patch 2 and inside the region 24 this side is covered as well, except for an area in the region 24 contacting a microneedle platform 210. Alternatively, only some parts of the skin-side may be covered, and e.g. the adhesive layer 23 only cover a part of the surface outside the region 24, for example only the skin-side under the stiffening body 22.

The flexible sheet 20 is deformable to conform to the contour of the skin 3 of the subject, and more specifically curves, when the patch 2 is applied, to conform to the contour. In this example, the sheet 20 is deformable at least between the edge 201 thereof and the region 24. The flexible sheet 20 may e.g. bend under its own weight and be resiliently bendable, but preferably exhibits a fabric-like drape, and can comprise woven or non-woven fabric layers. The flexible sheet 20 may be elastically stretchable in its planar direction which facilitates curving the sheet over the contour. In the region 24, the sheet 20 is deformable to a lesser extent due to the stiffening body 22. In this example, the platform 211 is rigid and the microneedles do not change their orientation relative to the base 210. Thus, the sheet 20 cannot deform in the area where the platform 211 extends between the skin 3 and the sheet 20. However, if the platform 211 changes orientation the flexible sheet 20 will deform with the platform.

The flexible sheet further comprises a backing layer 202 at a non-skin side of the intermediate layer, i.e. the side opposite to adhesive layer 203 and facing away from the skin 3 when the patch is applied. The backing layer 202 may e.g. protect the layers below and provide wear-resistance during use of the patch 2. In this example, the backing layer 202 further serves to attach a stiffening body 22 to the flexible sheet 20, which is explained below in more detail. As shown, the stiffening body 22 is embedded in the sheet 20, and more specifically at the upper side covered by the backing layer 202 and at the bottom by the intermediate layer 201. In this example, the top-surface of the backing layer 202 forms the upper surface of the patch 2.

The sheet 20 may comprise more layers. For example, an absorbent layer may be present between the intermediate layer and the upper surface which is in fluid communication with the needles, such that bodily fluids are extracted, and substances sampled from the body are absorbed in the layer. Additionally, or alternatively, a reservoir layer may be present which contains substances to be delivered.

As can be seen in FIG. 33A, prior to use, the patch 2 may comprise a removable liner 25 which covers at least the tacking parts of the skin-adhesive surface 200. The removable liner has a, non-skin adhesive, exposed surface facing away from the skin-adhesive surface 200. In this example, the liner covers the microneedles 21 of the patch as well. The liner 25 thus allows to ensure that the microneedles are not contaminated with hazardous substances or micro-organisms prior to use. Shortly before applying the patch 2 to the skin, the liner 25 may be removed, to expose the adhesive surface 200 and, in this example, the microneedles 210

The microneedle patch 2 further has one, two or more microneedles 21 projecting from the skin-adhesive surface 203. When the patch is applied, the microneedles perforate the skin, allowing substances to be exchanged between the patch 2 and the skin 3. For example, pharmaceutically active ingredients or other substances may be administered through the skin barrier into the body or substances from the body collected into the patch through the skin barrier. The administered substances can e.g. dissolve into the surrounding skin tissue and diffuse to the microcirculation of the skin for example or penetrate deeper into the body.

Generally speaking, the patch may have any suitable type and number of microneedles. The microneedles may e.g. be solid, coated, hollow, bio-degradable or non-bio-degradable, or a mixture thereof. The microneedles may be non-degradable needles with the pharmaceutically active ingredient embedded therein, such as porous needles which release an ingredient from the pores or, of needles where the ingredient diffuses out of the needle into the skin tissue, just to give some examples. In this example, the microneedles 21 are part of a microneedle platform 210 and extend beyond the adhesive layer out of the sheet. The microneedle platform 210 comprises a microneedle array 213, in which in this example a plurality of microneedles 21 is arranged in rows and columns. This array allows to perforate a large area of the skin. As shown, the platform 210 comprises a rigid base 211 from which the microneedles 21 project.

The microneedles 21 may have any suitable length and diameter. For example, a diameter of several tens to several hundreds of micrometers and/or a length of several tens, a few hundreds to a few thousands of micrometers have found to be suitable. Since the microneedle is relatively small in diameter and length as compared to the conventional needles, the activation of the nociceptors in the skin 3 is reduced and preferably completely avoided. Thus, significant alleviation of pain experience by the subject can be obtained when dermally or transdermally administering drugs or taking samples from the subject. The physical damage to the skin is minimal as a consequence of the small dimensions of the needles. Preferably, the dimensions are such that, under non-occlusive conditions, the perforations close once the microneedles are removed within less than 2 days, preferably less than 1 day, such as in less than 10 hours, for example in several hours. The perforations may be micropores, e.g. of a diameter less than 500 micrometer.

The microneedles 21 may be made of any material suitable for the application, such as silicon, metal, polymer, glass and ceramic, and in a variety of shapes and sizes. The microneedles may be manufactured using any technique suitable for the specific material, shape and size. For example, microfabrication techniques of adding, removing, and copying microstructures utilizing photolithographic processes, silicon etching, laser cutting, metal electroplating, metal electropolishing and micro-moulding may be used.

As mentioned, the patch 2 is provided with a stiffening body 22 attached to the flexible sheet 20. The stiffening body 22 stiffens the patch 2 in at least a parallel direction parallel to the skin-adhesive surface in the region 24 of the skin-adhesive surface which includes the microneedle 21. Thereby, the stiffening body 22 shields when applied to the skin, at least partially, the microneedle 21 from shear forces, e.g. caused by the subject moving or e.g. clothing rubbing over the patch. This allows to better fixate the microneedles 21. In addition, this ensures a more uniform insertion of the microneedles, and reduces e.g. complications in dosing due to natural variations in morphology of the patient's skin, where non-uniform insertion can inadvertently induce sheer stress and cause transverse bending of the microneedle structures.

In this example, the stiffening body 22 inhibits a change in orientation of the microneedle relative to the skin-adhesive surface in the region. More specifically, the stiffening body 22 limits the freedom of movement of the microneedle relative to the skin-adhesive surface because the base layer 201 extends over the microneedle 21 and the bendability of the base layer 22 is limited or removed by the stiffening body 22. The stiffening body 22 and the region 24 thus act as a stable base for the microneedles 21.

Such shear forces may also result from stretched skin bouncing back when the patch is applied to, and the microneedle has perforated, stretched skin. In such a case, as in the example, the stiffening body 22 can be oriented to shield the region 24 from a compressive force parallel to the skin-adhesive surface 200 exerted, when the patch is attached to the skin, on the flexible sheet 20 in reaction to stretching the skin. More specifically, the skin-adhesive surface 200 is attachable to a stretched area of the skin 3, and the stiffening body 22 is arranged to at least partially inhibit relaxation of the stretched skin in a covered part of the stretched area which is covered by the region. Also, the stiffening 22 can maintain the stretched skin 3 stretched underneath in the region 24, which allows to keep the perforations made with the patch open. Thereby the exchange of substances between the patch and the body of the subject can be improved. Additionally, this allows to avoid the relaxing skin to push the microneedles out of the perforations.

The shown examples of patches are attached when the skin 3 has already been stretched, or is being stretched, separately by a separate element outside the patch. Thus, the stretching of the skin 3 is not caused by the patch. Accordingly, the force with which the patch penetrates the skin 3 can well controlled, since this is not coupled to the stretching force.

In the examples of FIGS. 32,33 the stiffening body is resiliently deformable and when applied on the stretched skin 3 is either in its natural state, i.e. not resiliently deformed, or in a resiliently deformed, unstable state in which, absent any opposing external forces acting from outside the patch on the stiffening body, the stiffening body will return to its natural state. The relaxation forces exerted by the skin 3 are partially absorbed by a resilient deformation of the stiffening body which comes into an equilibrium where the stiffening body exerts on the skin 3 a spring force, equal in magnitude and compensating for but opposite to, the relaxation force the skin 3 exerts on the stiffening body 2.

In this example, the part of the skin-side surface underneath the region 24 is provided with adhesive, at least over the part corresponding to the perimeter of the region but the adhesive may also be present over the interior defined by the perimeter. Accordingly, when the patch 2 is attached, the region 24 itself will be tacked to the corresponding part of the skin contacting that part. Accordingly, the freedom of movement of the stretched skin is limited to that of the region 24, and accordingly relaxation of the stretched skin in the part can be prevented. This allows to maintain the perforations made with the patch open and thus ensure a good, e.g. dermal or transdermal, exchange of substances between the patch 2 and the skin 3 and/or to avoid the microneedles to be pushed out of the skin 3 by the relaxation of the skin 3.

The stiffening body 22 in this example is resiliently deformable, e.g. in one, two or more dimensions. Thus, the stiffening body 22 can adapt, to a lesser extent than the sheet 20, to the shape of the skin 3 where the patch 2 is applied and absorb and resist forces acting on the region 24. In the shown example for instance, the stiffening body 22 is flat absent any external forces acting thereon and can be resiliently stretched and compressed in any direction in the initial plane defined by the stiffening body 22 and be twisted or bend out of that initial plane. The stiffening body 22 can for example exhibit one, two or more of: elastically bendable, elastically compressible, elastically stretchable. In a currently preferred example, the stiffening body 22 is made of a silicone, rubber-like polymer material for instance, and preferably a solid body.

Although the stiffening body 24 may e.g. extend over a part of the boundary of the region 24 only, e.g. only an arc segment or a side thereof, in this example the stiffening body encloses the region 24. The stiffening body 22 inhibits at least partially wrinkling of the skin-adhesive surface in the region and in this example the shown stiffening body 22 is loop-shaped, and more specifically a, closed, uninterrupted loop around the region 24. The stiffening body 22 does not cover the region 24, but alternatively the stiffening body may cover the region 24 and e.g. be a disk or otherwise shaped plate instead a loop. The stiffening body 24 in FIG. 32 is shaped as a circular annulus but it will be apparent that the stiffening body 24 may e.g. be a rectangular, elliptical or other shape. Also, in this example, the stiffening body 24 is uninterrupted but the stiffening body 24 may alternatively be implemented with interruptions and e.g. be a closed, interrupted loop around the region of be implemented as unconnected bars, each defining a respective side of geometrical shape, for instance. Also, the stiffening body may be an open loop, and e.g. be C or U-shaped, in which case the region 24 is defined by the area enclosed by the open loop and an imaginary line connecting the unconnected ends of the open loop.

The stiffening body 22 may be located at any position on the sheet 20 suitable to shield the region 24. In the shown example, the stiffening body 22 is located at a distance from an edge 210 of the sheet 20. Thus, the part of the sheet between the edge 210 and the stiffening body 24 is not stiffened. Accordingly, the patch 2 can be still be applied on curved or moving parts of the body of the subject while at the same time securing the orientation of the microneedles 21.

In this example, the stiffening body also stiffens the patch 2 in the region 24 in a perpendicular direction, perpendicular to the skin-adhesive surface 200. The stiffening body stiffens the patch in the region such that a force towards the skin exerted on the patch, such as by the adhesive, as indicated with the arrows in FIG. 33 B is transferred in the region via the patch 2 to the microneedle 21 to maintain penetration in at least, and preferably through, the stratum corneum and optionally, further into the epidermis, e.g. deeper into the skin, until into the dermis or into the hypodermis subcutis. For example, the microneedles can for example have penetrated the dermis until into the papillary dermis or until into the reticular dermis. The microneedles may e.g. have pierced the stratum corneum, and any intermediate layers, until into one of the following skin layers without having pierced that layer: stratum lucidum, stratum granulosum, stratum spinonsum, stratum basale, basement membrane, papillary dermis, reticular dermis. Preferably, but not necessarily, the penetration of the microneedle(s) has not activated of the nociceptors in the skin.

When the patch 2 is attached to the skin 3, the microneedles 21 will receive a counterpressure from the skin 3 in a direction perpendicular to, and away from, the surface, especially where the skin has been stretched. The microneedles 21 transfer this pressure on the sheet 20, while the adhesive exerts a force in the direction perpendicular to, but towards the surface. By means of the stiffening in the region, the adhesive force can be effectively transferred onto the microneedles 21 to counteract the counter pressure.

As explained in more detail with reference to FIG. 37, the patch 2 can for example be stiffened in the region 24 by the stiffening body 22, in addition to shielding the region 24 from the forces, stretch and tension the sheet 20 in the region, e.g. similar to a drum skin when the patch is applied. The sheet may e.g. be not-tensioned initially and be tensioned when the patch 2 is applied to the skin. More specific, the counter pressure by the skin pushes the microneedle upwards while the adhesive layer pulls the sheet down, towards the skin. These opposite forces lead to the sheet 20 being tensioned and pushing the microneedles 21 towards the skin 3. Thereby, the microneedles 21 are retained into a penetrating position in the skin 3. In such a case, the stiffening body retains the flexible sheet 20 in the region 24 to resist a deformation caused by a counterpressure from the skin, counter to the pressure exerted by the microneedle 21 on the skin 3. In the shown example, for instance, the flexible sheet 20 spans the region, but in an un-used state of the patch is not tensioned.

The patch 2 may be applied and brought from the unapplied state illustrated in FIG. 33A into the applied state B by, if present, removing the liner 25 to expose the adhesive surface 200. Subsequently, the patch may be brought onto the skin with the adhesive surface 200 contacting the skin 3. Upon this contact, the patch 2 is tacked onto the skin by the stiction of the adhesive surface 200. Shortly before, during or after the adhesive surface 200 contacts the skin 3, the microneedle 21 penetrates the skin 3. In the example of FIG. 33 B, the skin 3 has been stretched and the microneedle penetrates the stretched skin3. Since in this example the surface under the region 24 contacting the skin is provided with adhesive, this region 24 is not only shielded against forces, but the freedom of movement of the stretched skin 3 underneath the region is coupled to the freedom of movement of the region 24. The stiffening body 22 as a consequence prevents the stretched skin from relaxation. This not only ensures that the perforations are kept open and hence the exchange of substances between the patch and the skin is maintained, but further allows to reduce the damaging effect of the microneedles prising the perforations can be reduced as well.

FIG. 34 shows several implementations of the microneedle patch. In the examples A and B of FIG. 34, the applied patch 2 has an open region through which the skin 3 is exposed. The stiffening body in this example shields the open region 24. More specifically, although the open region may be larger or smaller than this area, this open region 24 corresponds to the area of the skin where the exposed skin 3 has been penetrated with microneedles. After penetration, the microneedles have been removed, leaving the skin 3 with corresponding holes or perforations 32 and the perforated area exposed. Although other types of microneedles could be removed as well, in the example A, the microneedles are solid needles which simply perforate the skin 3. As shown, after perforation of the skin the microneedles are removed but the perforations 32 remain for a certain period of time. During this period, e.g. substances can be administered by applying a gel or cream on the skin 3 in the perforated area.

In example A, the microneedles are removed from the applied patch 2 while the remaining parts of the patch 2 are kept in place on the skin 3. The stiffening body 22 thus inhibits relaxation of the stretched skin 3 in the shielded, open region 24. Thus, undesired closure of the perforations 32 due to the relaxation can be reduced, and the conditions of applying substances can be more controlled.

In the example B of FIG. 34, the microneedles have been provided with a coating 28 with a pharmaceutically active ingredient which is released into the skin 3 after perforation thereof. Depending on the specific implementation, such a coating 28 may adhere to the walls of the holes 32 created in the skin 3, and thus be transferred from the patch into the skin tissue. This allows a continued release while the skin heals, or alternatively the microneedles may be kept in place until the coating has released the desired amount of pharmaceutically active ingredient. The stiffening body 22 inhibits relaxation of the stretched skin, which prevents undesired movement of the substances released from the coating and/or the transferred coating, e.g. due to repetitive stretching and relaxation of the skin 3 under the patch 2 when the subject moves body parts.

In the example C, the patch 2 is provided with microneedles made of a bio-degradable material, for instance soluble into the skin tissue, with a pharmaceutically active ingredient embedded therein. The ingredient may e.g. by embedded in a matrix but likewise inside the microneedle a separate reservoir filled with a formulation containing the pharmaceutically active ingredient may have been provided.

The microneedles are not actively removed but bio-degrade. When the microneedles dissolve into the skin tissue or otherwise bio-degrade after application of the patch 2, the pharmaceutically active ingredient embedded in the microneedles is released. Since the stiffening body 22 shields the region 24, the microneedles are shielded from forces and accordingly the circumstances under which they degrade can be more controlled.

In the example D of FIG. 34, the microneedles 21 of the patch 2 remain in the skin during the treatment and are not removed from the patch 2. The patch 2 is provided with a fluid transport system 29 which comprises a reservoir 290 e.g. for a substance to be administered to or collected from the subject. The microneedle 21 of the patch 2 are implemented as a base with one or more projecting needles inside which channels 291 are present. Through the channels 291 a substance can be administered from the reservoir 290. Alternatively, the reservoir 290 may initially be empty and be used to store a substance collected from the subject. For example, a substance may be collected transdermally from the subject, e.g. by applying the patch during a predetermined period of time and subsequently removing the patch 2 from the skin 3, where during the period a bodily fluid flows through the channels 291 into the reservoir 290. In this example, the stiffening body maintains the skin stretched around the microneedles stretched and thus ensures that the fluid-transport conditions in the skin, and more specific the flow resistivity, are maintained to allow the fluid to flow from or towards the microneedles. It will be apparent that in the example D, instead of the rerserveroir 290, e.g. swellable microneedles may be used which swell with interstitial fluid when applied into the skin and may be taken from the skin 3 for e.g. further analysis.

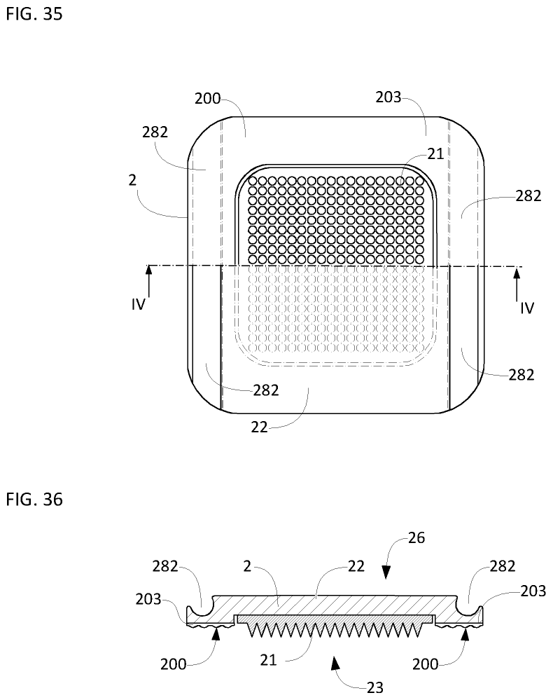

Referring to FIGS. 35-36, another example of a microneedle patch 2 is shown therein. The shown example may be used in an applicator 1 as described above, but it will be apparent that the applicator can use other patches and, vice versa the patches may be applied in another manner on the skin of a subject.

In this example, the stiffening body 22 is a rigid plate which extends over the microneedle(s). In the example of FIGS. 35-36 the relaxation forces exerted by the skin 3 create a compressive force on the rigid plate, which is resisted by the rigid plate without significant, or in an example even noticeable, deformation of the rigid plate. This allows to use the region 24 as a platform for highly sensitive elements, such as electronic circuitry and boards. Alternatively, the plate can e.g. be an elastically or plastically bendable plate, which can be bent to conform to the contour of the skin 3 when the patch is applied. Although not shown, the plate can e.g. house electronics such as sensor, a reservoir for substances to be administered or sampled, or other elements. It will be apparent to one skilled in the art, that such element may alternatively or additionally be mounted on the top side, opposite to the skin-facing side, of the plate for example. As shown, the plate is attachable to the skin by an adhesive surface 200. Like the example of FIGS. 32-33, the adhesive surface 200 encloses a microneedle platform with microneedles 21. The skin-adhesive surface 200 covers the entire skin-side 23 of the patch 2 except for the microneedle platform and is. However, it will be apparent that alternatively, only some parts of the skin-adhesive surface 200 may be provide with the adhesive and the adhesive layer 203 only cover a part of the skin-side 23. If the patch 2 is applied to a stretched skin, the attachment between the plate and the skin 3 at opposite sides of the microneedle(s) inhibits relaxation of the skin 3 in the penetrated area, and accordingly the conditions of administering or sampling substances are more controlled.

The plate further extends over the microneedles. More specific in this example, in the area of the microneedles the skin-side of the plate is covered by the microneedle platform. The plate, when the patch is attached, limits the freedom of movement of the microneedles in a direction away from the skin 3, i.e. perpendicular to the adhesive surface 200 at skin-side 23. Thereby, e.g. rotating or tilting movement of the microneedles are reduced and accordingly an inadvertent widening of the perforations thereby and undesired damage to the skin can be reduced.

In this example, the plate projects, in a direction parallel to the skin-side 23, over the microneedles and the skin-adhesive layer 203 is provided at the skin-side of the projecting parts of the plate. Thus, the adhesive surface 200 extends under the projecting parts of the plate.

Although in this example, the upper surface 26 is formed by the exposed top side of the plate, it will be apparent that the plate may, locally or completely, be covered with additional layers.

As shown, in this example, the patch 2 is provided at the upper surface 26 with respective recesses 282, in this figure shaped as elongated slots which extend at opposite sides of the microneedles 21. The recesses 282 are open at the top side and in the recess a corresponding projection of the platform with a shape conforming to the recess 282 can be admitted to establish a releasable fixation of the patch on the platform 18, as e.g. illustrated in FIG. 30.

Referring to FIG. 37, in this example the flexible sheet 20 comprises an elastic layer 204 which is stretched by the stiffening body 22, and which extends over the microneedle 21. As shown, in this example, between the elastic layer 204 and the microneedle, in this example a resilient block 205 is present, which can be compressed in a direction from the elastic layer 204 to the microneedle 21 and thus exert a pressure on the microneedle 21 towards the skin 3. As illustrated in this example with the downwards arrows, the stiffening body 22 is attached to the skin 3 and holds thereon with a force towards the skin 3 which it exerts on the on the layer 204. The microneedles 21 exerts a force on the layer 204 in the opposite direction at another locations, as illustrated with the upwards arrows. As a consequence of these opposite forces, the layer 204 is tensioned and exerts a counter force on the microneedles corresponding to the force holding the stiffening body 22. This counter force pushes the microneedles into the skin and thus maintains them in penetration.

### Kit

Referring to FIG.39, the example of a kit 4 shown therein comprises an applicator 1 and a patch 2. As shown in this FIG. 39, the applicator in the kit may be applicator 1 as described in this application and/or the patch be as described in this application, but it will be apparent that one of the applicator or the patch may be a different, e.g. as an applicator as described in this application on which a conventional microneedle patch is mounted.

In this example, the microneedle patch 2 is pre-mounted in the holder 17 of the application but alternatively the microneedle patch 2 may be provided separately, e.g. in a package containing several patches and a single applicator for example. In such a case, the microneedle patches 2 may be separately, and optionally individually, packaged. The kit 4 can further comprise a package 5 in which the applicator and patch are provided, and e.g. of a suitable packaging material such as plastic. The package 5 may be a sterile package and alternatively or additionally be provided with instructions as to how to use and place the applicator on a skin, as well as with instructions of treatment of a condition such as dosing and frequency of application for a specific condition. In this example, for instance, the package 5 is a sterile sealed bag of a suitable material, such as plastic.

FIG. 38 shows a package for the patches 2. In this example, a tray 50 with recesses 51 is shown. In each recess 51 a patch 2 is present, in this example pre-mounted on an insert 123 such as suitable for the example of FIGS. 19-26. The patches 2 are oriented with the microneedles to the closed side of the recesses 51 and the recesses 51 have a shape conforming to the inserts 123 (or alternatively to the patches 2). The edges of the recesses 51 thus seal off together with the inserts or patches, the microneedles and thus allow to maintain those sterile. To take a respective patch, the applicator may be placed on the tray, with the platform 18 or the base-body over the recess 51. The platform of base body may then clamp or otherwise attach to the patch or insert and be moved away from the tray, taking the patch out of the recess.

In the shown example, the left hand patches are provided with a marking to allow to differentiate the patch to be taken. The shown marking is part of an orientation dependent coupling, and the base 10 may be provided with a corresponding part that couples to the marking and provided with a movement mechanism that changes the position of the corresponding part relative to the rest of the base, such that the user can, when correctly holding the applicator, only take the patch of which the marking has the orientation corresponding to that of the corresponding part.

The kit 4 may be used to apply the microneedle patch to the skin of a subject. In such as case, for example, prior to applying the microneedle patch 2, the skin may have been treated with alcohol and/or hair removed, such as by shaving, in the area where the microneedle patch 2 is to be applied. In case the kit 4 is provided in a package 5, the microneedle patch 2 and applicator 1 may be taken out of the package. If not already mounted in the holder, the microneedle patch 2 may then be mounted in the holder. If present, the liner 25 may be removed prior or after mounting the microneedle patch 2 in the holder 12. Thereafter, the applicator 1 may be placed on the skin 3.

The applicator 1 may e.g. be placed on a part of the body of the subject selected from the group: head, ear, neck, limb, arm, upper arm, lower arm, hand, leg, upper leg, lower leg, foot, torso, chest, abdomen, pelvic region, back, shoulders, buttocks. For example, the applicator may be placed on the inside of the lower arm. Thereby, a relatively low amount of force is needed to penetrate the skin, since the skin is relatively thin in that area, and additionally few preparations are required because this body part has not that much hair. The applicator may be adapted to the thickness of the skin of the selected body part, and e.g. to exert more force on the microneedle patch if the applicator is for a part with relatively thick skin layers, such as at a buttock, compared to the force of an applicator for a part with relatively thin skin layers, such as an ear. It will be apparent that, e.g. in case of self-administration, the body part is preferably within reach of the hands of the subject.

The skin can then be stretched by the applicator and the microneedle patch applied on the stretched skin by the applicator, e.g. as described above with reference to the examples of FIGS. 1-30. That is, the applicator brings the microneedle patch 2 onto the skin with the adhesive surface 200 contacting the skin 3. Upon this contact, the microneedle patch 2 is tacked onto the skin by the stiction of the adhesive surface 200. Shortly before, during or after the adhesive surface 200 contacts the skin 3, the microneedle 21 penetrates the skin 3.

The applicator 1 may when the microneedle is applied, for example provide haptic, more specific tactile, feedback at predetermined points of the process of applying the patch. For example, the point can be one or more of: the stretching of the skin reached a predetermined threshold, the microneedle 21 penetrates the skin 3, the pressure exerted on the skin 3 by the patch 2 and platform 18 as reached a predetermined threshold, the patch 2 can be separated from the platform 18. In the examples, for instance, the applicator 1 provides feedback that the patch 2 can be separated from the platform 18 due to the snap-fit connection 183,193 locking. However, other feedback to the user or the subject may be provided, such as by the breaking of the bond 137, to indicate the point in time the skin 3 has been stretched sufficiently, or the plate 195 moving or the upright element 185 projecting out of the contact surface 17 to indicated that the pressure exerted has reached the desired amount, for example.

The microneedle patch 2 applied on the skin 3 may then be used to exchange substances between the microneedle patch and the body of the subject through the area of the skin perforated by the microneedle(s). In case, as in this example, the microneedle patch 2 has a stiffening part and the microneedle patch 2 is adhered to the skin 3 in at least the edge of the area shielded by the stiffening part 22, the stiffening part in the microneedle patch 2 not only retains the shape of the microneedle patch 2 in the shielded area by also inhibits relaxation of the stretched skin in that area. Since the skin 3 remains stretched even after the applicator 1 has been separated from the microneedle patch 2 and has been removed from the skin 3, the perforations are kept open and the exchange of substances can be improved.

In the foregoing specification, the invention has been described with reference to specific examples of embodiments of the invention. It will, however, be evident that various modifications and changes may be made therein without departing from the broader scope of the invention as set forth in the appended claims.

For example, it will be apparent that where in the examples microneedles are used, this may be a single microneedle or multiple microneedles. For example, the patch may be provided with several tens of microneedles.

Furthermore, it will be apparent that the applicator may be provided with a control which allows to set the pressure of force with which the microneedle patch is applied, e.g. to

Also, it will be apparent that the coupling may comprise other types of releasable latches with a latching state in which the latch latches movement of the holder induced by the force exerted by an (e.g. biased) actuator, and a released state in which the latch allows the movement instead of the form-closed connection in the example of FIGS. 1 and 2. For example, an electro-magnetic latch may be used which is controlled by an electronic circuit connected to a sensor which senses the distance between the contact parts. The electronic circuit may then e.g. release the latch when the distance exceeds a predetermined release distance for instance.

Also, although in the examples an exchange of substances between the patch and the skin has been described, via the perforations, other types of interaction between the skin and the patch may be established. For example, characteristics of the penetrated layers or of the body below the penetrated layers may be modified, e.g. by heating the perforated area with the microneedle, or properties of the skin or the body be sensed, e.g. through resistive measurements.

It will be apparent for example that the applicator can be a single-use applicator, which once the microneedle patch is applied is not re-used, and optionally made such that a new patch cannot be mounted on the applicator. Alternatively, the applicator may be a re-usable applicator.

Furthermore, it will be apparent that in the example the releasable fixations, connections etc are non-destructively releasable unless specified otherwise.

For example, the microneedle patch may be applied by a medical practitioner or be used to self-administer by a subject. In this respect, the subject can be a human or an animal. The microneedle patch may e.g. be applied on a part of the body of the subject selected from the group: head, ear, neck, limb, arm, upper arm, lower arm, hand, leg, upper leg, lower leg, foot, torso, chest, abdomen, pelvic region, back, shoulders, buttocks. For example, the patch may be applied to the inside of the lower arm. Thereby, a relatively low amount of force is needed to penetrate the skin, since the skin is relatively thin in that area, and additionally few preparations are required because this body part has not that much hair. The applicator may be adapted to the thickness of the skin of the selected body part, and e.g. to exert more force on the microneedle patch if the applicator is for a part with relatively thick skin layers, such as at a buttock, compared to the force of an applicator for a part with relatively thin skin layers, such as an ear. It will be apparent that, e.g. in case of self-administration, the body part is preferably within reach of the hands of the subject.

Also, it will be apparent that instead of tactile feedback or signals other haptic feedback or signals, and generally other for humans perceptible feedback or signals may be provided.

For example, where a movement of an object is described (e.g. relative to another object) it will be apparent that this is a relative movement, and accordingly depending on the chosen reference base-body, the object may be moving relative to an observer while the other object is static, the other object may be moving while the object is static relative to the observer or both objects may be moving relative to the observer but in different directions and/or with different speeds.

In this respect, the term “form-closed” refers to the German term “Formschluss”, which is a connection between at least two connected elements formed by the interlocking shapes of the elements and in which the absence of a connecting force does not release the connection. In other words, in the case of a form-closed connection, the shapes of the connected elements are in the way of the other, such that the connection cannot be released without deforming the shapes. Likewise, the term “force-closed” refers to the German term “Kraftschluss”, which is a connection between at least two connected surfaces formed by a connecting force perpendicular to the connecting surfaces, in which the absence of the connecting force releases the connection.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements The mere fact that certain measures are recited in mutually different claims does not indicate that a combination of these measures cannot be used to advantage.

1. A re-usable applicator for applying a microneedle patch to a skin of a subject, comprising:
a base for positioning onto the skin, the base comprising:
a skin-side end;
a holder for holding the microneedle patch in position relative to the skin-side end, and
at least two contact parts at the skin-side end for contacting the skin, of which at least one is movable over the skin and away from another contact part to stretch the skin at least during penetration of the skin by a microneedle of the patch;
the applicator further comprising:
an interface for an actuator, for actuating a movement of the microneedle patch relative to the skin-side end to penetrate at least into the stratum corneum of the epidermis of the skin with the microneedle.

2. The applicator of claim 1, wherein the actuator is a mechanical actuator connected to the interface for actuating a movement of the microneedle patch.

3.-4. (canceled)

5. The applicator claim 1, comprising a coupling which couples the movement of the patch towards the skin-side end to a movement of the contact part over the skin, for penetrating the skin by the microneedle when the skin is stretched a predetermined amount.

6. The applicator of claim 5, wherein the coupling is between the contact parts and the actuator, for triggering actuation of the movement of the patch when the contact part is moved a predetermined distance away from the other contact part, such as upon reaching the predetermined distance or thereafter.

7. (canceled)

8. The applicator of claim 1, wherein the coupling is arranged to trigger actuation of the movement of the patch when the patch is at a distance from the skin.

9. The applicator of claim 1, wherein the coupling is arranged to trigger actuation of the movement of the patch when a predetermined part of the applicator other than the contact parts, is in contact the skin after the separation between contact parts reaching the predetermined distance.

10.-12. (canceled)

13. The applicator of claim 1, comprising a contact surface for a hand of the human operator, and wherein the coupling couples the contact parts to the contact surface for actuating movement of the contact parts away from each other as reaction to a force exerted by the hand on the contact surface.

14.-15. (canceled)

16. The applicator of claim 1, wherein the contact parts are separate parts each having a fixed proximal end connected to the base and a distal, free-end with a contact surface for contacting the skin, the free-ends of the contact parts being movable relative to each other in at least a direction parallel to the skin.

17.-18. (canceled)

19. The applicator claim 1, wherein the holder comprises:
a movable platform, movable relative to the skin-side end in a direction towards the skin, for placing the microneedle patch oriented with a skin-adhesive surface facing the skin-side end, and
a base-body for holding the movable platform in position relative to the base.

20. The applicator of claim 19, wherein the platform is admitted with the microneedle patch, when placed on the platform, facing the skin-side end, in a space in the base-body, the space having an open-side facing the skin-side end through which the platform can pass, to allow the patch to move from an initial position up to or beyond the skin-side end.

21.-26. (canceled)

27. The applicator claim 1, wherein the holder comprises a guide for guiding the movement of the patch along a predetermined path between the distant position and the skin contacting position.

28. (canceled)

29. The applicator of claim 1, wherein the actuator is arranged to control the movement of the patch to have a velocity upon contacting the skin by the microneedle patch sufficient to penetrate the skin by impact.

30. (canceled)

31. The applicator claim 1, wherein
the actuator comprises a spring arranged to be biased, the spring coupled to the holder, for exerting on the holder, when biased, a force towards the skin-side end; and
the coupling comprises:
a releasable latch with a latching state in which the latch latches movement of the holder induced by the force and a released state in which the latch allows the movement, and
a control engaging on the releasable latch to control the state of the releasable latch to enter into the released state when the contact parts are moved the predetermined distance away from each other.

32. The applicator of claim 31, wherein
the holder comprises:
a movable platform, movable relative to the skin-side end in a direction towards the skin, for placing the microneedle patch oriented with a skin-adhesive surface facing the skin-side end, and
a base-body for holding the movable platform in position relative to the base; and
the spring is arranged between the platform and the base-body to exert a force from the base-body to the platform.

33. (canceled)

34. The applicator claim 1, comprising a latch which latches movement of the holder in a direction away from the base at a predetermined point after movement has started, such as upon or after penetration of the skin.

35.-39. (canceled)

40. The applicator of claim 1, wherein the base comprises:
a housing with a bore extending towards the holder;
a kinetic transducer for converting a movement of the housing relative to the holder into potential energy;
a potential energy storage for storing the potential energy;
a release for releasing the potential energy; and
a kinetic transducer for converting the stored potential energy into a force on the platform upon release.

41.-43. (canceled)

44. The applicator of claim 1, for penetrating through the stratum corneum further into the epidermis until into, or through, one of the group selected from: the stratum lucidum, the stratum granulosum, the stratum spinonsum, the stratum basale, basement membrane.

45. A microneedle patch for use in an applicator in a re-usable applicator to apply the microneedle patch to a skin of a subject, the applicator comprising:
a base for positioning onto the skin, the base comprising:
a skin-side end;
a holder for holding the microneedle patch in position relative to the skin-side end, and
at least two contact parts at the skin-side end for contacting the skin, of which at least one is movable over the skin and away from another contact part to stretch the skin at least during penetration of the skin by a microneedle of the patch;
the applicator further comprising an interface for an actuator, for actuating a movement of the microneedle patch relative to the skin-side end to penetrate at least into the stratum corneum of the epidermis of the skin with the microneedle;
the microneedle patch comprising:
a skin-adhesive surface for attaching the patch to the skin of a subject;
a microneedle platform with microneedles projecting from the skin-adhesive surface out of the patch; and
a stiffening body extending over the microneedles which, in a region of the skin-adhesive surface which includes the microneedle, stiffens the patch in at least a parallel direction parallel to the skin-adhesive surface,
the microneedle platform covering a part of a skin-side of the stiffening body and the skin-adhesive surface enclosing at least a part of the part of the skin-side not covered by the microneedle platform.

46.-68. (canceled)

69. The applicator of claim 1, with the patch mounted in the holder or provided separately.

70.-73. (canceled)

74. A method of applying a microneedle patch to a skin of a subject with a reusable applicator, the applicator comprising:
a base for positioning onto the skin, the base comprising:
a skin-side end;
a holder for holding the microneedle patch in position relative to the skin-side end, and
at least two contact parts at the skin-side end for contacting the skin, of which at least one is movable over the skin and away from another contact part to stretch the skin at least during penetration of the skin by a microneedle of the patch;
the applicator further comprising an interface for an actuator, for actuating a movement of the microneedle patch relative to the skin-side end to penetrate at least into the stratum corneum of the epidermis of the skin with the microneedle;
the method comprising:
positioning the applicator provided with the microneedle patch on the skin;
stretching the skin by moving at least one contact part over the skin and away from another contact part;
actuating a movement of the microneedle patch relative to the skin; and
penetrating at least into the stratum corneum of the epidermis of the skin with the microneedle by the movement of the microneedle patch.

75.-84. (canceled)
